(19) United States
(12) Patent Application Publication
(10) Pub. No.: US 2019/0008957 A1
(43) Pub. Date: Jan. 10, 2019

(54) COMBINATION THERAPIES INCLUDING INHIBITORS OF THE EXTRACELLULAR DOMAIN OF E-CADHERIN

(71) Applicant: The Research Foundation for The State of New York, Albany, NY (US)

(72) Inventors: Sabine Brouxhon, Stony Brook, NY (US); Stephanos Kyrkandies, Stony Brook, NY (US)

(21) Appl. No.: 15/882,513

(22) Filed: Jan. 29, 2018

Related U.S. Application Data
(63) Continuation of application No. 14/385,351, filed on Sep. 15, 2014, now abandoned.
(60) Provisional application No. 61/736,475, filed on Dec. 12, 2012.
(60) Provisional application No. 61/611,390, filed on Mar. 15, 2012.

(51) Int. Cl. A61K 39/395; A61K 45/06; C07K 16/30

(52) U.S. Cl. CPC A61K 39/3955; A61K 2039/505; C07K 16/30; A61K 45/06

(57) ABSTRACT

The present invention is based on our work with E-cadherin, including soluble portions of this integral membrane glycoprotein. The compositions of the present invention include therapeutically effective amounts of a first agent that targets epitopes within one or more of the EC2-EC5 subdomains of the ectodomain of E-cadherin (including these domains in the shed sEcad fragment) and a second agent that inhibits one or more of: endothelial tube formation; angiogenesis; the human epidermal growth factor receptor family (i.e. HER1-4); an insulin-like growth factor 1 receptor (IGF-1R); any other receptor tyrosine kinase receptor family member; the P13K-MAPK pathway; and the P13K/Akt/mTOR pathway. The compositions can be used in the treatment of epithelial cancers and can be used to inhibit tumor growth and metastasis. The invention also features methods in which cancer is staged.

### CROSS-REFERENCE TO RELATED APPLICATIONS

This application is a continuation of U.S. application Ser. No. 14/385,651, which was filed on Sep. 15, 2014, which is a U.S. National Stage of International Application No. PCT/US2013/032656, which was filed on Mar. 15, 2013, which claims the benefit of the filing date of U.S. Provisional Application No. 61/611,390, which was filed Mar. 15, 2012, and of the filing date of U.S. Provisional Application No. 61/736,475, which was filed Dec. 12, 2012. The content of these prior applications is hereby incorporated by reference in its entirety.

### STATEMENT REGARDING FEDERALLY FUNDED RESEARCH

This invention was made with government support under Grant Numbers CA133910 and ES015832 awarded by the National Institutes of Health. The government has certain rights in the invention.

### FIELD OF THE INVENTION

The compositions and methods of the present invention are related to targeting both sEcad, which is a soluble portion of the extracellular domain of the cell-cell adhesion protein E-cadherin, and a cellular receptor or intracellular effector in the MAPK-PI3K/Akt/mTOR, src and inhibitor of apoptosis (IAP/XIAP, survivin, livin, c-IAP-1, or c-IAP-2) signaling pathways.

### BACKGROUND

E-cadherin is an integral transmembrane glycoprotein that helps maintain epithelial cell-cell adhesion. Loss of E-cadherin function (full length) has been demonstrated to result in cellular de-differentiation, proliferation and increased invasiveness in cancers of the skin, lung, stomach, intestine and breast (Brouxhon et al., Cancer Res. 67(16):7654-7664, 2007; Hirohashi, Am. J. Pathol. 153(2):333-339, 1998; Chen et al., Cancer Lett. 201:97-106, 2003). Moreover, loss of E-cadherin staining in biopsy specimens from breast cancer patients has been associated with a poor prognosis and short metastasis-free periods (Pederson et al., Brit. J. Cancer 87:1281-1286, 2002).

The full length protein is composed of an extracellular domain consisting of five subdomains designated EC1-EC5, a single transmembrane region, and a cytoplasmic domain (see Shiraishi et al., J. Immunol. 175(2):1014-1021, 2005). The EC1 subdomain, which is the most distant from the cell membrane surface, contains a histidine-alanine-valine (HAV) triplet found in cadherin-expressing cells (including E-, N-, P-, and R-cadherin-expressing cells), and this subdomain is thought to be essential for promoting the cell-cell contact mediated by E-cadherin (Beavon, European J. Cancer 36:1607-1620, 2000).

Full-length E-cadherin contains a cleavage site for various proteases near the transmembrane domain, and cleavage at that site produces a soluble N-terminal peptide of ˜80-84 kDa called soluble E-cadherin (sEcad). Shedding of sEcad occurs constitutively at low levels in normal, unstimulated epithelial cells and at elevated levels in patients with epithelial-derived tumors such as breast, skin, lung, prostate, gastric and colorectal cancers (Banks et al., J. Clin. Pathol. 48:179-180, 1995; Baranwal et al., Biochem. Biophys. Res. Corn. 384(1):6-11, 2009; Chan et al., Gut 48:808-811, 2001; Charalabopoulos et al., Exp. Oncol. 28(1):83-85, 2006; Kuefer et al., Clin. Cancer Res. 9:6447-6452, 2003; Shirahama et al., J. Dermatol. Sci. 13:30-36, 1996; Velikova et al., Br. J. Cancer 77:1857-1863, 1998). Shedding of sEcad has also been reported to increase in normal, non-cancerous canine kidney cells after the induction of apoptosis (Steinhusen et al., J. Biol. Chem. 276:4972-4980, 2001).

While sEcad levels are increased in the urine or sera of cancer patients and are also elevated when normal cells undergo apoptosis, the biologic activity of this shed protein is not well understood. A number of studies have demonstrated that sEcad disrupts normal epithelial cell-cell adhesion, induces epithelial cell scattering, and enhances tumor cell proliferation, migration, and invasion (Gil et al., Gynecol. Oncol. 108(2):361-369, 2008; Maretzky et al., Proc. Natl. Acad. Sci. USA 102(26):9182-9187, 2005; Marambaud et al., EMBO J. 21(8):1948-1956, 2002; Najy et al., J. Biol. Chem. 283(26):18393-18401, 2008; Noe et al., J. Cell Sci. 114:111-118, 2001; Ryniers et al., Biol. Chem. 383:159-165, 2002; and Symowicz et al., Cancer Res. 67(5):2030-2039, 2007). The signaling pathways modulating these biologic functions are still unclear. Studies using the SKBr3 breast cancer cell line demonstrated that sEcad-HER2 complexes were induced by exogenous addition of a purified extracellular fusion protein (Fc-sEcad), leading to extracellular signal-regulated kinase (ERK) activation (Najy et al., J. Biol. Chem. 283(26):18393-18401, 2008). The human EGF receptor belongs to the ErbB or HER family of receptor tyrosine kinases, which are overexpressed or dysregulated in many epithelial tumors (Mendelsohn and Baselga, Oncogene 19:6550-6565, 2000; Burgess, Growth Factors 26:263-274, 2008). This family of receptors activates downstream signaling molecules such as ERK, which in turn activates a range of cancer cell behaviors including cell proliferation, migration, invasion and angiogenesis (Hanahan and Weinberg, Cell 100:57-70, 2000; Shields et al., Trends Cell Biol. 10:147-154, 2000). Accordingly, a number of anti-EGF therapies have been developed. These include small molecule tyrosine kinase inhibitors, monoclonal antibodies, and cancer vaccines (Fukuoka et al., Proc. Am. Soc. Clin. Oncol. 21:292a Abs1188, 2002; Lage et al., Ann. Med. 35:327-336 (2003); Mateo et al., Immunotechnology 3:71-81, 1997; Slamon et al., N. Engl. J. Med. 344:783-792, 2001; and Yu et al., J. Clin. Invest. 110:289-294, 2002). These therapeutic strategies are limited in that only some tumors, at a defined maturation stage, express the specific receptor/antigen and not all tumors with a certain histology and stage overexpress the target receptor/antigen (only 20% to 50% of breast cancers overexpress the EGF receptor). Thus, response rates for these types of drugs remains low (Mendelson and Baselga, Oncogene 19:6550-6565, 2000; Ortega et al., Cancer Control 17(1):7-15, 2010). In addition, tumors initially responsive to these drugs eventually develop acquired resistance (Jackman et al., Clin. Cancer Res. 12:3908-3914, 2006; Ortega et al., Cancer Control 17(1):7-15, 2010; and Riely et al., Clin. Cancer Res. 12:839-844, 2006).

### SUMMARY

The present invention is based on our work with E-cadherin, including both membrane-bound and soluble portions of this integral membrane glycoprotein. More specifically, the compositions and methods described herein relate, at least in part, to our studies demonstrating that soluble E-cadherin modulates receptor tyrosine kinases, mitogen-activated protein kinases (MAPKs), and P13K/Akt/mTOR signaling. In a first aspect, the compositions of the present invention include therapeutically effective amounts of a first agent that specifically targets one or more of the second, third, fourth, or fifth subdomains (EC2, EC3, EC4 and EC5, respectively (collectively, EC2-EC5)) of the E-cadherin ectodomain, or of such subdomains in a soluble E-cadherin (sEcad) fragment thereof, but does not specifically target the first subdomain (EC1) of E-cadherin or sEcad. These sEcad-inhibitors can be combined with, or used in conjunction with, a second agent that inhibits one or more of: endothelial tube formation; angiogenesis; an insulin-like growth factor receptor (e.g., IGF-1R); a vascular endothelial growth factor (VEGF) receptor (VEGFR (e.g., bevacizumab)); a transforming growth factor receptor (e.g., TGFB-I/II)); vimentin/vimentin shedding; src; a receptor in the ErbB family of receptor tyrosine kinases (e.g., HER1 (with the provisio that cetuximab and panitumumab are excluded), HER2 (with the provisio that trastuzumab is excluded), HER3, and HER4); other receptor tyrosine kinases (e.g. a receptor in the PDGF, FGF, HGF, RET, Axl, KLG, DDR, LTK, ROR, Tie, Trk, RYK, Eph, or MuSK receptor families (particularly receptors that signal through the MAPK-PI3K/Akt/mTOR signaling pathway)); src; the Ras-Raf-MEK-ERK pathway; and the PI3K/Akt/mTOR pathway. The amounts of the first and second agents, taken together, are therapeutically effective. In particular embodiments, the first agent specifically targets EC4 and/or EC5; specifically targets EC4; or specifically targets EC5.

In the present pharmaceutical compositions, the first agent or the second agent can be a protein scaffold, and the scaffold can be an antibody. For example, when the first agent is an antibody, it can be an antibody or a biologically active fragment thereof that specifically binds an epitope comprising amino acid residues in one or more of the EC2, EC3, EC4 or EC5 subdomains of the E-cadherin ectodomain or the sEcad fragment thereof, but not in the EC1 subdomain of E-cadherin or sEcad. Whether acting as the first agent or the second agent, the antibody can be a chimeric, humanized, or human antibody, a single chain antibody, or a monoclonal or polyclonal antibody, and any antibody or antibody type can be conjugated to a drug to form an antibody-drug conjugate. The antibody can be of the immunoglobulin G (IgG) class or the immunoglobulin M (IgM) class. Any of the antibodies can be detectably labeled. It is to be understood that wherever an antibody can be used as described herein, a biologically active variant of that antibody can also be used.

The second agent can be a protein scaffold that specifically binds a receptor tyrosine kinase. For example, the second agent can be an antibody, as broadly described herein, including biologically active fragments thereof, that inhibits HER1, HER2, HER3, and/or HER4 in any combination of these receptors or other receptor tyrosine kinases. Thus, the protein scaffold can be a pan-HER inhibitor. Although the protein scaffolds are not limited to those that exert their action through any particular mechanism, the scaffolds can inhibit the dimerization of HER2. More specifically, the second agent can be pertuzumab, erlotinib, gefitinib, lapatinib, cetuximab or canertinib or biologically active variants thereof.

The first and/or second agents can also be nucleic acids or small molecules (e.g., a small organic compound) that inhibit a target as described herein (e.g., a receptor tyrosine kinase such as HER1, HER2, HER3, HER4, VEGFR, IGFR (e.g., IGF-1R), FGF, HGF, PDGF, Eph, and/or any of the other targets described herein and known in the art. In one embodiment, the invention features a pharmaceutical composition comprising a first agent and a second agent in which the first agent specifically targets one or more of the second, third, fourth, or fifth subdomains (EC2, EC3, EC4 and EC5, respectively) of the ectodomain of E-cadherin or the soluble E-cadherin (sEcad) fragment thereof, but does not target the first subdomain (EC1) of the E-cadherin ectodomain or of sEcad, and the second agent is ziv-aflibercept, vandetanib, AG1024, or NVP-ADW742. As in any other dual-agent composition of the invention, the amounts of the first and second agents, taken together, can be therapeutically effective.

Instead of administering first or second agents that specifically bind an E-cadherin subdomain as described herein, one can also administer an agent that elicits the production of such agents (antibodies) within a given patient. For example, the invention features pharmaceutical compositions that include a first agent and a second agent, where the first agent is an antigenic polypeptide including an amino acid sequence from one or more of the EC2-EC5 subdomains of the E-cadherin ectodomain or the soluble E-cadherin (sEcad) fragment thereof (to, as in other embodiments, the exclusion of the EC1 subdomain). Alternatively, the first agent can be an antigenically active fragment or other variant of these polypeptides or an expression vector comprising a nucleic acid sequence encoding the antigenic polypeptide or the antigenically active fragment or other variant thereof. The second agent can be any of those described herein. For example, the second agent can be ziv-aflibercept, vandetanib, AG1024, or NVP-ADW742. In other embodiments, the second agent can inhibit an effector in the MAPK intracellular signaling pathway or the PI3K/Akt/mTOR signaling pathway.

In any of the present compositions, the first agent or the second agent can be detectably labeled (allowing for both therapeutic and diagnostic or prognostic use). In any of the present compositions, the second agent can be the MEK inhibitor GDC-0973; an ERK inhibitor; the PI3K inhibitor GDC-0941, GSK1059615, BKM120, or GDC0941; the Akt inhibitor perifosine or MK2206; the mTOR inhibitor temsirolimus, everolimus, rapamycin, or AZD8055; the p70S6K inhibitor LY2584702; the PI3K/mTOR inhibitor NVP-BEZ235; or the p70S6K/Akt inhibitor LY2780301.

Any of the pharmaceutical compositions described herein can be formulated such that the composition kills malignant E-cadherin-expressing cells but does not kill non-malignant cells to any appreciable extent. While the invention is not limited by its mechanism of action, the death of malignant E-cadherin-expressing cells may be induced by programmed cell death, growth arrest, anoikis, necrosis, or autophagy. In some instances, the compositions can induce senescence in malignant E-cadherin-expressing cells or inhibit metastases.

Any of the pharmaceutical compositions described herein can be free of cytotoxic amounts of any carrier, diluent, or excipient. Similarly, by way of exclusion, any given agent listed herein can be explicitly excluded from a given composition. For example, where the inventors teach that a composition can include A, B, or C, it is to be understood that the composition can include, for example, A and B to the exclusion of C.

The pharmaceutical compositions described herein can be delivered in a pharmaceutical formulation that: (a) produces, upon administration to a patient, a serum level of the first agent of about 1-50 mg/kg (e.g., about 1-8 mg/kg), or (b) produces, upon addition to a cell culture, a concentration of the first agent of about 1-500 μg/mL of cell culture medium (e.g., about 200-400 μg/mL). The amounts of the first and second agents, taken together, are therapeutically effective.

The compositions can be used in the treatment of epithelial cancers and may effectively inhibit cellular proliferation, migration, and/or invasiveness. Thus, the present compositions and methods can be used to inhibit tumor growth and metastasis (e.g., by inhibiting cellular proliferation, migration, and/or invasiveness). The present compositions and methods can be used in the treatment of “triple negative” breast cancer patients (in whom breast cancer cells test negative for estrogen receptors, progesterone receptors, and HER2). In addition to therapeutic and prophylactic treatment methods, the invention features methods in which cancer is staged depending on the relative amounts of full-length (FL) E-cadherin and soluble E-cadherin (sEcad). The greater the amount of sEcad relative to the amount of FL E-cadherin, the more advanced the cancer.

The methods in which anti-E-cadherin antibodies are administered encompass dose-specific therapies, and the therapies can be selectively directed toward and cytotoxic for breast, lung, GI tract (e.g. esophagus, stomach, intestine, colon), pancreas, bladder, prostate, skin, oral and head and neck cancers, other epithelial cancers and cancers of tissues derived from the ectoderm (e.g., the central nervous system, the lens of the eye, cranial and sensory ganglia and nerves, and connective tissue in the head). The therapeutic and prophylactic methods described herein can be carried out in connection with other cytotoxic therapies (e.g., chemotherapy, hormone therapy, radiotherapy, and small-molecule inhibitors, antibody-based therapies (e.g., monoclonal anti-EGF antibody therapy)).

More specifically, the invention features methods of treating cancer by administering to a patient in need of treatment a pharmaceutical composition as described herein. The cancer can be one within an epithelialized tissue. The cancer can be a cancer of the alimentary canal (e.g., the mouth, throat, esophagus, stomach, intestine, colon, rectum or anus), central nervous system, breast, skin (e.g., squamous cell carcinoma or melanoma), reproductive system (cervical cancer, uterine cancer, ovarian cancer, vulval or labial cancer, prostate cancer, testicular cancer, or cancer of the male genital tract), lung, or urinary tract. Any of the methods can include a step of providing a biological sample from the patient and determining whether the sample includes an elevated level of sEcad and/or another predictive biomarker for cancer. The biological sample can be a urine, saliva, cerebrospinal fluid, blood, stool, or biopsy sample, and this step can be carried out before administering the pharmaceutical composition. An elevated level of sEcad and/or another predictive biomarker for cancer indicates that the patient is a good candidate for the treatment. This evaluative step can also be carried out at one or more times after administering the pharmaceutical composition, with a reduced level of sEcad and/or another predictive biomarker for cancer indicating that the patient is responding well to the treatment.

As noted throughout, the compositions of the present invention can include more than one therapeutic agent. For example, an agent that inhibits sEcad (e.g., anti-sEcad antibodies) may be administered with another therapeutic agent, such as a cytotoxic agent or cancer chemotherapeutic (many of which are specifically described herein). In the pharmaceutical compositions of the invention, the first and second agents can be physically combined in a single dosage form. However, any combination therapy can also be delivered by methods in which the patient receives two dosage forms (one containing a first therapeutic agent and one containing a second therapeutic agent). Thus, in the methods of the invention, two or more therapeutic agents may be variously delivered. In some cases, a patient may receive two or more therapeutic agents simultaneously. In some cases, a patient may receive concurrent administration of two or more therapeutic agents; concurrent administration does not require that the agents be administered at the same time or by the same route, as long as there is an overlap in the time period during which the agents are exerting their therapeutic effect. Sequential administration is also encompassed by the present methods, in which case, in a treatment regime, a patient is treated with a first agent and, upon completion with the first agent, is treated with a second agent. Administration of the two agents can be separated by days, weeks, or months. Thus, the invention encompasses not only compositions containing first and second agents (any of the first agents and any of the second agents described herein), but also kits including first and second agents for a combined therapy (whether simultaneous, overlapping, or sequential), and the methods of the invention likewise encompass treatment with two or more agents simultaneously, in an overlapping manner, or sequentially.

In a particular aspect, the invention features a pharmaceutical composition that includes a first agent and a second agent, wherein: the first agent is an antigenic polypeptide comprising an amino acid sequence from one or more of the EC2-EC5 subdomains of the ectodomain of E-cadherin, including these ectodomains in the shed sEcad fragment, but not the EC1 subdomain, or an antigenically active fragment or other variant thereof, or an expression vector comprising a nucleic acid sequence encoding the antigenic polypeptide or the antigenically active fragment or other variant thereof; the second agent inhibits HER1 (with the provisio that cetuximab and panitumumab are excluded); inhibits HER2 (with the provisio that trastuzumab is excluded); inhibits HER3; inhibits HER4; inhibits a vascular endothelial growth factor receptor (VEGFR); or inhibits an insulin-like growth factor receptor (IGFR); or inhibits any other receptor of the tyrosine kinase family of receptors that may or may not signal through the PI3K/Akt/mTOR signaling pathway.

### BRIEF DESCRIPTION OF THE DRAWINGS

FIG. 1A shows the amino acid sequence of human E-cadherin (SEQ ID NO:1), with the extracellular subdomains EC2-EC5 indicated by alternating underlining (EC2 and EC4 are underlined with double lines, and EC3 and EC5 are underlined with single lines).

FIG. 1B is a schematic representation of a wild type human E-cadherin with sEcad as indicated. The length of the soluble fragment can vary and may terminate in the 4^{th} or 5^{th} extracellular domain or, in other cases, in the transmembrane domain (see, for example, the study by Noe et al., J. Cell Sci. 114(1):111-118, 2000).

### DETAILED DESCRIPTION

As described further below, we tested the effectiveness of a variety of commercially available monoclonal and polyclonal antibodies targeting the extracellular domain of E-cadherin, including the DECMA-1 antibody employed by Espada et al. (J. Cell Physiol. 219:84-93, 2009), Fouquet et al. (J. Biol. Chem. 279(41):43061-43069, 2004) and Galaz et al. (J. Cell Physiol. 205(1):86-96, 2005) in a panel of both epithelial cancer cells and non-cancerous cells. When we repeated these experiments, we found that application of this antibody at concentrations as low as 40 μg/mL surprisingly induced cell death in both cancer cells (i.e., MCF-7, SCC12b, SCC13, CRL-1555, PAM212, SP308 and KLN205 cells) as well as in non-cancerous cells that were employed as controls (i.e., human breast epithelial cells and PHK and PMK cells). Moreover, treatment of these cancer and non-cancerous cells with the control IgG isotype antibody at the same concentrations (40 μg/mL) also induced the same level of cell death in both types of cells. These data suggest a non-specific induction of cell death after the application of the antibody. In our laboratory, applying a more dilute solution of the antibody targeting the extracellular domains EC2-EC5 of E-cadherin (a low dose of 10-20 μg/mL) induced cell death, apparently by apoptosis, in cancer cells only. We observed no untoward effects on non-cancerous cells. Moreover, application of the control IgG isotype to non-cancerous cells at a concentration of 10-20 μg/mL had no detectable effect on cell viability in general. These concentrations between 10-20 μg/mL are ˜20-50 times lower than the concentration used by Espada et al. (J. Cell Physiol. 219:84-93, 2009), Fouquet et al. (J. Biol. Chem. 279(41):43061-43069, 2004) and Galaz et al. (J. Cell Physiol. 205(1):86-96 (2005)). Accordingly, it is our expectation that the present pharmaceutical formulations and preparations can be made and used as low dose formulations (e.g., at doses lower than those suggested by Espada and others in prior studies), and such formulations are encompassed by the present invention. While one of ordinary skill in the art will appreciate that dosage can vary based on a number of considerations, the compositions and methods of the present invention can be directed to dosages of dual-agent compositions in which a desired effect is observed with respect to cancer cells while non-cancerous cells remain unaffected or not substantially affected. As described above, exogenous application of 10-20 μg/mL of antibodies (monoclonal or polyclonal) against the EC2-EC5 subdomains of E-cadherin selectively killed a representative panel of human and mouse tumor cell lines. These concentrations may be useful in the present compositions and methods or, at least, will serve to help identify clinically useful formulations of a “low dose” character. We also have data showing that such antibodies are cytotoxic to the HT29 human colon cell line, the NCI-H292 human lung cell line, and the KLN205 murine lung cancer cell line. Accordingly, the present compositions and methods can be directed to the treatment of patients with colon or lung cancer (two prevalent and devastating types of cancer). Moreover, we demonstrated that non-cancerous cells, including normal human breast epithelial cells, normal human and mouse keratinocytes, mouse 3T3 fibroblasts and human endothelial cells remained unaffected in our analyses using antibodies that target the subdomains at low concentrations. The molecular pathways by which targeting varying combinations of the EC2-EC5 extracellular domains of E-cadherin in epithelial-derived tumor cells induces cell death has yet to be elucidated. However, we demonstrated that dying cancer cells upregulated the pro-apoptotic marker p53; we observed this upregulation in the MCF-7 breast cancer, mouse SCC and mouse KLN205 lung cancer cell lines after treatment with antibody against the EC2-EC5 E-cadherin domains.

As noted above, the compositions of the invention include agents that specifically target one or more of EC2, EC3, EC4 and EC5 domains of the extracellular domain of E-cadherin (i.e. EC2-5), including the shed sEcad fragment (EC2-5) (herein referred to as sEcad). These agents do not, however, specifically target the EC1 subdomain. The agents may subsequently inhibit sEcad, or they may bind, inhibit, or sequester another target, such as a cell survival receptor, in the tumor cell microenvironment. While the present compositions are not limited to those that exert their effect by any particular mechanism, our working hypothesis is the agents of the invention interfere with the ability of sEcad to provide signals beneficial to cancer cells. For example, we hypothesize that cancer cells secrete sEcad into the microenvironment where it provides a functional scaffold that mimics normal cell-to-cell contact. Thus, actually or effectively removing sEcad from the tumor microenvironment perturbs the ability of tumor cells to remain adherent and survive. In other instances, an agent of the invention may inhibit sEcad activity by binding to an epitope on sEcad that interacts with another cellular target (e.g., HER-2), thereby altering downstream signaling events involved in cell survival, cell proliferation, cell migration and/or invasion. Alternatively, or in addition, an agent may not bind a specific epitope required for sEcad signalling but may instead bind sEcad in a way that sequesters, tags, or targets it for destruction, thereby lowering its concentration in the tumor microenvironment and rendering it unavailable to mimic cell-cell interactions or binding to cellular receptors. For example, the agents and compositions may reduce sEcad shedding by, for example, binding to E-cadherin and blocking the cleavage site or otherwise blocking or inhibiting the release of sEcad. Thus, a composition or agent, as described herein, can specifically target one or more of the EC2-EC5 subdomains, effectively preventing sEcad from providing one or more of the signals it otherwise would by interfering with a specific epitope or actually reducing sEcad levels.

For ease of reading, we may refer to an agent that specifically targets amino acid residues in one or more of the EC2-EC5 subdomains of sEcad but not in the first subdomain (EC1) of sEcad more simply as a targeting agent. The targeting agents of the invention can be a protein scaffold, such as a modified fibronectin domain or an immunoglobulin, or a fragment or other variant thereof, that specifically binds to amino acid residues in one or more of the EC2-EC5 subdomains of E-cadherin and shed sEcad (but not to the EC1 domain). Where the agent is, or includes, a protein (e.g., a protein scaffold or antigenic polypeptide), we may refer to the agent as a protein-based therapeutic. We tend to use the term “protein” to refer to longer amino acid polymers, and we tend to use the term “polypeptide” to refer to shorter sequences or to a chain of amino acid residues within a larger molecule or complex. Both terms, however, are meant to describe an entity of two or more subunit amino acids, amino acid analogs, or other peptidomimetics, regardless of post-translational modification (e.g., amidation, phosphorylation or glycosylation). The subunit amino acid residues can be linked by peptide bonds or other bonds such as, for example, ester or ether bonds. The terms “amino acid” and “amino acid residue” refer to natural and/or non-natural or synthetic amino acids, which may be D- or L-form optical isomers.

The anti-sEcad antibodies can assume various configurations and encompass proteins consisting of one or more polypeptides substantially encoded by immunoglobulin genes. Any one of a variety of antibody structures can be used, including an intact antibody, antibody multimers, or antibody fragments or other variants thereof that include functional, antigen-binding regions of the antibody. We may use the term “immunoglobulin” synonymously with “antibody.” The antibodies may be monoclonal or polyclonal in origin. Regardless of the source of the antibody, suitable antibodies include intact antibodies, for example, IgG tetramers having two heavy (H) chains and two light (L) chains, single chain antibodies, chimeric antibodies, humanized antibodies, complementary determining region (CDR)-grafted antibodies as well as antibody fragments, e.g., Fab, Fab′, F(ab′)2, scFv, Fv, and recombinant antibodies derived from such fragments, e.g., camelbodies, microantibodies, diabodies and bispecific antibodies.

An intact antibody is one that comprises an antigen-binding variable region (V_{H} and V_{L}) as well as a light chain constant domain (C_{L}) and heavy chain constant domains, C_{H1}, C_{H2} and C_{H3}. The constant domains may be native sequence constant domains (e.g. human native sequence constant domains) or amino acid sequence variants thereof. As is well known in the art, the V_{H} and V_{L} regions are further subdivided into regions of hypervariability, termed “complementarity determining regions” (CDRs), interspersed with the more conserved framework regions (FRs). The extent of the FRs and CDRs has been defined (see, Kabat et al. Sequences of Proteins of Immunological Interest, Fifth Edition, U.S. Department of Health and Human Services, NIH Publication No. 91-3242, 1991, and Chothia, et al., J. Mol. Biol. 196:901-917 (1987). The CDR of an antibody typically includes amino acid sequences that together define the binding affinity and specificity of the natural Fv region of a native immunoglobulin binding site.

An anti-sEcad antibody can be from any class of immunoglobulin, for example, IgA, IgG, IgE, IgD, IgM (as well as subtypes thereof (e.g., IgG₁, IgG₂, IgG₃, and IgG₄)), and the light chains of the immunoglobulin may be of types kappa or lambda. The recognized human immunoglobulin genes include the kappa, lambda, alpha (IgA₁ and IgA₂), gamma (IgG₁, IgG₂, IgG₃, IgG₄), delta, epsilon, and mu constant region genes, as well as the myriad immunoglobulin variable region genes.

The term “antigen-binding portion” of an immunoglobulin or antibody refers generally to a portion of an immunoglobulin that specifically binds to a target, in this case, an epitope comprising amino acid residues within or between one or more of the second to fifth subdomains of sEcad (e.g., within or between the fourth and fifth subdomains). An antigen-binding portion of an immunoglobulin is therefore a molecule in which one or more immunoglobulin chains are not full length, but which specifically binds to a cellular target. Examples of antigen-binding portions or fragments include: (i) an Fab fragment, a monovalent fragment consisting of the VLC, VHC, CL and C_{H1} domains; (ii) a F(ab′)₂ fragment, a bivalent fragment comprising two Fab fragments linked by a disulfide bridge at the hinge region; (iii) a Fv fragment consisting of the VLC and VHC domains of a single arm of an antibody, and (v) an isolated CDR having sufficient framework to specifically bind, e.g., an antigen binding portion of a variable region. An antigen-binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see e.g., Bird et al., Science 242:423-426 (1988); and Huston et al., Proc. Natl. Acad. Sci. USA 85:5879-5883 (1988)). Such scFvs can be a target agent of the present invention and are encompassed by the term “antigen-binding portion” of an antibody.

An “Fv” fragment is the minimum antibody fragment that contains a complete antigen-recognition and binding site. This region consists of a dimer of one heavy chain and one light chain variable domain in tight, con-covalent association. It is in this configuration that three hypervariable regions of each variable domain interact to define an antigen-binding site on the surface of the V_{H}-V_{L} dimer. While six hypervariable regions confer antigen-binding specificity, even a single variable domain (or half of an Fv comprising only three hypervariable regions specific for an antigen) has the ability to recognize and bind antigen, although at a lower affinity than the entire binding site. To improve stability, the VH-VL domains may be connected by a flexible peptide linker such as (Gly₄Ser)₃ to form a single chain Fv or scFV antibody fragment or may be engineered to form a disulfide bond by introducing two cysteine residues in the framework regions to yield a disulfide stabilized Fv (dsFv).

As noted, other useful antibody formats include diabodies, minibodies and bispecific antibodies. A diabody is a homodimer of scFvs that are covalently linked by a short peptide linker (about 5 amino acids or less). By using a linker that is too short to allow pairing between two domains on the same chain, the domains can be forced to pair with the complementary domains of another chain and create two antigen-binding sites (see, e.g., EP 404,097 and WO 93/11161 for additional information regarding diabodies). A diabody variant, (dsFv)₂ or a linear antibody useful in the present compositions and methods includes a pair of tandem Fd segments (V_{H}-C_{H1}-V_{H}-C_{H1}) that form a pair of antigen binding regions (see, e.g., Zapata et al., Prot. Eng. 8:1057 (1995)). Useful minibodies are homodimers of scFv-CH₃ fusion proteins. In the minibody variant, the Flex minibody, the scFv is fused to the hinge region of IgG, which is in turn, linked to the CH₃ region by a 10-amino acid linker.

A bispecific antibody, which recognizes two different epitopes, can also be used as long as one arm specifically binds sEcad as described herein. A variety of different bispecific antibody formats have been developed. For example, useful bispecific antibodies can be quadromas, i.e., an intact antibody in which each H-L pair is derived from a different antibody. Typically, quadromas are produced by fusion of two different B cell hybridomas, followed by screening of the fused calls to select those that have maintained the expression of both sets of clonotype immunoglobulin genes. Alternatively, a bispecific antibody can be a recombinant antibody. Exemplary formats for bispecific antibodies include, but are not limited to tandem scFvs in which two single chains of different specificity are connected via a peptide linker; diabodies and single chain diabodies.

Fragments of antibodies are suitable for use in the methods provided so long as they retain the desired specificity of the full-length antibody and/or sufficient specificity to inhibit cancer cell survival, proliferation, or metastasis. Thus, a fragment of an anti-sEcad antibodies, as described herein, can retain the ability of the intact antibody to bind to the recited subdomains. These antibody portions can be obtained using conventional techniques known to one of ordinary skill in the art, and the portions can be screened for utility in the same manner as intact antibodies are screened as anti-cancer agents.

Methods for preparing antibody fragments are well known in the art and encompass both biochemical methods (e.g. proteolytic digestion of intact antibodies which may be followed by chemical cross-linking) and recombinant DNA-based methods in which immunoglobulin sequences are genetically engineered to direct the synthesis of the desired fragments. Exemplary biochemical methods are described in U.S. Pat. Nos. 5,855,866; 5,877,289; 5,965,132; 6,093,399; 6,261,535; and 6,004,555. Nucleic acids encoding a chimeric or humanized chain can be expressed to produce a contiguous polypeptide. See, e.g., Cabilly et al., U.S. Pat. No. 4,816,567; Cabilly et al., European Patent No. 0,125,023 BI; Boss et al., U.S. Pat. No. 4,816,397; Boss et al., European Patent No. 0,120,694 BI; Neuberger et al., WO 86/01533; Neuberger et al., European Patent No. 0,194,276 BI; Winter, U.S. Pat. No. 5,225,539; and Winter, European Patent No. 0,239,400 BI. See also, Newman et al., BioTechnology 10:1455-1460 (1992), regarding CDR-grafted antibodies and Ladner et al. (U.S. Pat. No. 4,946,778) and Bird et al., Science 242:423-426 (1988)) regarding single chain antibodies.

Antibody fragments can be obtained by proteolysis of the whole immunoglobulin by the non-specific thiolprotease, papain. Papain digestion yields two identical antigen-binding fragments, termed “Fab fragments,” each with a single antigen-binding site, and a residual “Fc fragment.” The various fractions can be separated by protein A-Sepharose or ion exchange chromatography. The usual procedure for preparation of F(ab′)₂ fragments from IgG of rabbit and human origin is limited proteolysis by the enzyme pepsin. Pepsin treatment of intact antibodies yields an F(ab′)₂ fragment that has two antigen-combining sites and is still capable of cross-linking antigen. A Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. F(ab′)₂ antibody fragments were originally produced as pairs of Fab′ fragments that have hinge cysteines between them. Other chemical couplings of antibody fragments are known.

Also within the scope of the present invention are methods of making a targeting agent (e.g., an antibody or an antigen-binding fragment or other variant thereof) that targets sEcad by, for example, specifically binding to the second, third, fourth or fifth subdomain of sEcad (or to an epitope including amino acid residues in two or more of these subdomains). For example, variable regions can be constructed using PCR mutagenesis methods to alter DNA sequences encoding an immunoglobulin chain (e.g., using methods employed to generate humanized immunoglobulins; see e.g., Kanunan et al., Nucl. Acids Res. 17:5404, 1989; Sato et al., Cancer Research 53:851-856, 1993; Daugherty et al., Nucleic Acids Res. 19(9):2471-2476, 1991; and Lewis and Crowe, Gene 101:297-302, 1991). Using these or other suitable methods, variants can also be readily produced. For example, in one embodiment, cloned variable regions can be mutagenized, and sequences encoding variants with the desired specificity can be selected (e.g., from a phage library; see e.g., Krebber et al., U.S. Pat. No. 5,514,548; and Hoogenboom et al., WO 93/06213).

Other suitable methods of producing or isolating immunoglobulins that specifically recognize a cellular target as described herein include, for example, methods that rely upon immunization of transgenic animals (e.g., mice) capable of producing a full repertoire of human antibodies (see e.g., Jakobovits et al., Proc. Natl. Acad. Sci. USA 90:2551-2555, 1993; Jakobovits et al., Nature 362:255-258, 1993; Lonberg et al., U.S. Pat. No. 5,545,806; and Surani et al., U.S. Pat. No. 5,545,807).

As is well known in the art, monoclonal antibodies are homogeneous antibodies of identical antigenic specificity produced by a single clone of antibody-producing cells, and polyclonal antibodies generally recognize different epitopes on the same antigen and are produced by more than one clone of antibody producing cells. Each monoclonal antibody is directed against a single determinant on the antigen. The modifier, monoclonal, indicates the character of the antibody as being obtained from a substantially homogeneous population of antibodies, and is not to be construed as requiring production of the antibody by any particular method. For example, the monoclonal antibodies may be made by the hybridoma method first described by Kohler et al. (Nature 256:495, 1975) or by recombinant DNA methods (see, e.g., U.S. Pat. No. 4,816,567). The monoclonal antibodies may also be isolated from phage antibody libraries using the techniques described in Clackson et al. (Nature 352:624-628, 1991) and Marks et al. (J. Mol. Biol. 222:581-597, 1991), for example.

The monoclonal antibodies herein can include chimeric antibodies, i.e., antibodies that typically have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855, 1984). Chimeric antibodies of interest include primatized antibodies comprising variable domain antigen-binding sequences derived from a non-human primate (e.g. apes, Old World monkeys, New World monkeys, prosimians) and human constant region sequences.

Various methods for generating monoclonal antibodies (mAbs) are well known in the art. See, e.g., the methods described in U.S. Pat. No. 4,196,265, incorporated herein by reference. The most standard monoclonal antibody generation techniques generally begin along the same lines as those for preparing polyclonal antibodies (Antibodies: A Laboratory Manual, Cold Spring Harbor Laboratory Press, Cold Spring Harbor, N.Y. (1988)). Typically, a suitable animal can be immunized with a selected immunogen to stimulate antibody-producing cells. Rodents such as mice and rats are exemplary animals, although rabbits, sheep, frogs, and chickens can also be used. Mice can be particularly useful (e.g., BALB/c mice are routinely used and generally give a higher percentage of stable fusions).

Following immunization, somatic cells with the potential for producing the desired antibodies, specifically B lymphocytes (B cells), can be selected for use in MAb generation and fusion with cells of an immortal myeloma cell, generally one of the same species as the animal that was immunized. Myeloma cell lines suited for use in hybridoma-producing fusion procedures typically are non-antibody-producing, have high fusion efficiency, and enzyme deficiencies that render then incapable of growing in certain selective media which support the growth of only the desired fused cells (hybridomas). Any one of a number of myeloma cells can be used, as are known to those of skill in the art. For example, where the immunized animal is a mouse, one can use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one can use R210.RCY3, Y3-Ag 1.2.3, IR983F, 4B210 or one of the above listed mouse cell lines. U-266, GM1500-GRG2, LICR-LON-HMy2 and UC729-6, all can be useful in connection with human cell fusions.

This culturing can provide a population of hybridomas from which specific hybridomas can be selected, followed by serial dilution and cloning into individual antibody producing lines, which can be propagated indefinitely for production of antibody.

Methods for producing monoclonal antibodies are well known in the art and can include purification steps. For example, the antibodies can generally can be further purified, for example, using filtration, centrifugation and various chromatographic methods, such as HPLC or affinity chromatography, all of which are techniques well known to one of ordinary skill in the art. These purification techniques each involve fractionation to separate the desired antibody from other components of a mixture. Analytical methods particularly suited to the preparation of antibodies include, for example, protein A-Sepharose and/or protein G-Sepharose chromatography.

The anti-sEcad antibodies of the invention may include CDRs from a human or non-human source. “Humanized” antibodies are generally chimeric or mutant monoclonal antibodies from mouse, rat, hamster, rabbit or other species, bearing human constant and/or variable region domains or specific changes. Techniques for generating a so-called “humanized” antibody are well known to one of ordinary skill in the art.

The framework of the immunoglobulin can be human, humanized, or non-human (e.g., a murine framework modified to decrease antigenicity in humans), or a synthetic framework (e.g., a consensus sequence). Humanized immunoglobulins are those in which the framework residues correspond to human germline sequences and the CDRs result from V(D)J recombination and somatic mutations. However, humanized immunoglobulins may also comprise amino acid residues not encoded in human germline immunoglobulin nucleic acid sequences (e.g., mutations introduced by random or site-specific mutagenesis ex vivo). It has been demonstrated that in vivo somatic mutation of human variable genes results in mutation of framework residues (see Nature Immunol. 2:537, 2001). Such an antibody would be termed “human” given its source, despite the framework mutations. Mouse antibody variable domains also contain somatic mutations in framework residues (See Sem. Immunol. 8:159, 1996). Consequently, transgenic mice containing the human Ig locus produce immunoglobulins that are commonly referred to as “fully human,” even though they possess an average of 4.5 framework mutations (Nature Genet. 15:146-56, 1997). Accepted usage therefore indicates that an antibody variable domain gene based on germline sequence but possessing framework mutations introduced by, for example, an in vivo somatic mutational process is termed “human.”

Humanized antibodies may be engineered by a variety of methods known in the art including, for example: (1) grafting the non-human complementarity determining regions (CDRs) onto a human framework and constant region (a process referred to in the art as humanizing), or, alternatively, (2) transplanting the entire non-human variable domains, but providing them with a human-like surface by replacement of surface residues (a process referred to in the art as veneering). Humanized antibodies can include both humanized and veneered antibodies. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783, 1992; Lonberg et al., Nature 368:856-859, 1994; Morrison, Nature 368:812-13, 1994; Fishwild et al., Nature Biotechnology 14:845-51, 1996; Neuberger, Nature Biotechnology 14:826, 1996; Lonberg and Huszar, Intern. Rev. Immunol. 13:65-93, 1995; Jones et al., Nature 321:522-525, 1986; Morrison et al., Proc. Natl. Acad. Sci, USA, 81:6851-6855, 1984; Morrison and Oi, Adv. Immunol., 44:65-92, 1988; Verhoeyer et al., Science 239:1534-1536, 1988; Padlan, Molec. Immun. 28:489-498, 1991; Padlan, Molec. Immunol. 31(3):169-217, 1994; and Kettleborough, C. A. et al., Protein Eng. 4(7):773-83, 1991).

In addition to chimeric and humanized antibodies, fully human antibodies can be derived from transgenic mice having human immunoglobulin genes (see, e.g., U.S. Pat. Nos. 6,075,181; 6,091,001; and 6,114,598), or from phage display libraries of human immunoglobulin genes (see, e.g. McCafferty et al., Nature 348:552-554, 1990; Clackson et al., Nature 352:624-628, 1991, and Marks et al., J. Mol. Biol. 222:581-597, 1991). In some embodiments, antibodies may be produced and identified by scFv-phage display libraries using standard methods known in the art.

The anti-sEcad antibodies may be modified to modulate their antigen binding affinity, their effector functions, or their pharmacokinetics. In particular, random mutations can be made in the CDRs and products screened to identify antibodies with higher affinities and/or higher specificities. Such mutagenesis and selection is routinely practiced in the antibody arts. A convenient way for generating such substitutional variants is affinity maturation using phage display.

CDR shuffling and implantation technologies can be used with the antibodies provided herein, for example. CDR shuffling inserts CDR sequences into a specific framework region (Jirholt et al., Gene 215:471 (1988)). CDR implantation techniques permit random combination of CDR sequences into a single master framework (Soderlind et al., Immunotechnol. 4:279, 1999; and Soderlind et al., Nature Biotechnol. 18:852, 2000). Using such techniques, CDR sequences of the anti-sEcad antibody, for example, can be mutagenized to create a plurality of different sequences, which can be incorporated into a scaffold sequence and the resultant antibody variants screened for desired characteristics, e.g., higher affinity. In some embodiments, sequences of the anti-sEcad antibody can be examined for the presence of T cell epitopes, as is known in the art. The underlying sequence can then be changed to remove T cell epitopes, i.e., to “deimmunize” the antibody.

Recombinant technology using, for example phagemid technology, allows for preparation of antibodies having a desired specificity from recombinant genes encoding a range of antibodies. Certain recombinant techniques involve isolation of antibody genes by immunological screening of combinatorial immunoglobulin phage expression libraries prepared from RNA isolated from spleen of an immunized animal (Morrison et al., Mt. Sinai J. Med. 53:175, 1986; Winter and Milstein, Nature 349:293, 1991; Barbas et al., Proc. Natl. Acad. Sci. USA 89:4457, 1992). For such methods, combinatorial immunoglobulin phagemid libraries can be prepared from RNA isolated from spleen of an immunized animal, and phagemids expressing appropriate antibodies can be selected by panning using cells expressing antigen and control cells. Advantage of this approach over conventional hybridoma techniques include approximately 10⁴ times as many antibodies can be produced and screened in a single round, and that new specificities can be generated by H and L chain combination, which can further increase the percentage of appropriate antibodies generated.

One method for the generation of a large repertoire of diverse antibody molecules in bacteria utilizes the bacteriophage lambda as the vector (Huse et al., Science 246:1275, 1989). Production of antibodies using the lambda vector involves the cloning of heavy and light chain populations of DNA sequences into separate starting vectors. Vectors subsequently can be randomly combined to form a single vector that directs co-expression of heavy and light chains to form antibody fragments. The general technique for filamentous phage display is described (U.S. Pat. No. 5,658,727). In a most general sense, the method provides a system for the simultaneous cloning and screening of pre-selected ligand-binding specificities from antibody gene repertoires using a single vector system. Screening of isolated members of the library for a pre-selected ligand-binding capacity allows the correlation of the binding capacity of an expressed antibody molecule with a convenient means to isolate a gene that encodes the member from the library. Additional methods for screening phagemid libraries are described (U.S. Pat. Nos. 5,580,717; 5,427,908; 5,403,484; and 5,223,409).

One method for the generation and screening of large libraries of wholly or partially synthetic antibody combining sites, or paratopes, utilizes display vectors derived from filamentous phage such as M13, fl or fd (U.S. Pat. No. 5,698,426, incorporated herein by reference). Filamentous phage display vectors, referred to as “phagemids,” yield large libraries of monoclonal antibodies having diverse and novel immunospecificities. The technology uses a filamentous phage coat protein membrane anchor domain as a means for linking gene-product and gene during the assembly stage of filamentous phage replication, and has been used for the cloning and expression of antibodies from combinatorial libraries (Kang et al., Proc. Natl. Acad. Sci. USA 88:4363, 1991; and Barbas et al., Proc. Natl. Acad. Sci. USA 88:7978, 1991). The surface expression library is screened for specific Fab fragments that bind neuraminidase molecules by standard affinity isolation procedures. The selected Fab fragments can be characterized by sequencing the nucleic acids encoding the polypeptides after amplification of the phage population.

One method for producing diverse libraries of antibodies and screening for desirable binding specificities is described (U.S. Pat. Nos. 5,667,988 and 5,759,817). The method involves the preparation of libraries of heterodimeric immunoglobulin molecules in the form of phagemid libraries using degenerate oligonucleotides and primer extension reactions to incorporate degeneracies into CDR regions of immunoglobulin variable heavy and light chain variable domains, and display of mutagenized polypeptides on the surface of the phagemid. Thereafter, the display protein is screened for the ability to bind to a preselected antigen. A further variation of this method for producing diverse libraries of antibodies and screening for desirable binding specificities is described U.S. Pat. No. 5,702,892, incorporated herein by reference). In this method, only heavy chain sequences are employed, heavy chain sequences are randomized at all nucleotide positions that encode either the CDRI or CDRIII hypervariable region, and the genetic variability in the CDRs can be generated independent of any biological process.

In addition to the combinatorial immunoglobulin phage expression libraries disclosed above, one molecular cloning approach is to prepare antibodies from transgenic mice containing human antibody libraries. Such techniques are described (U.S. Pat. No. 5,545,807, incorporated herein by reference). Such transgenic animals can be employed to produce human antibodies of a single isotype, more specifically an isotype that is essential for B cell maturation, such as IgM and possibly IgD. Another method for producing human antibodies is described in U.S. Pat. Nos. 5,545,806; 5,569,825; 5,625,126; 5,633,425; 5,661,016; and 5,770,429, wherein transgenic animals are described that are capable of switching from an isotype needed for B cell development to other isotypes.

The anti-sEcad immunoglobulins may be modified to reduce or abolish glycosylation. An immunoglobulin that lacks glycosylation may be an immunoglobulin that is not glycosylated at all; that is not fully glycosylated; or that is atypically glycosylated (i.e., the glycosylation pattern for the mutant differs from the glycosylation pattern of the corresponding wild type immunoglobulin). The IgG polypeptides include one or more (e.g., 1, 2, or 3 or more) mutations that attenuate glycosylation, i.e., mutations that result in an IgG CH2 domain that lacks glycosylation, or is not fully glycosylated or is atypicially glycosylated. Mutations of the asparagine residue at amino acid 297 in human IgG1 is an example of such a mutation. The oligosaccharide structure can also be modified, for example, by eliminating the fusose moiety from the N-linked glycan.

Antibodies can also be modified to increase their stability and or solubility in vivo by conjugation to non-protein polymers, e.g, polyethylene glycol. Any PEGylation method can be used as long as the anti-sEcad antibody retains the ability to selectively bind the second, third, fourth or fifth subdomain of sEcad.

A wide variety of antibody/immunoglobulin frameworks or scaffolds can be employed so long as the resulting polypeptide includes at least one binding region that is specific for the target, i.e., the second, third, fourth, or fifth subdomain of sEcad. Such frameworks or scaffolds include the five main idiotypes of human immunoglobulins, or fragments thereof (such as those disclosed elsewhere herein), and include immunoglobulins of other animal species, preferably having humanized aspects. Single heavy-chain antibodies such as those identified in camelids are of particular interest in this regard.

One can generate non-immunoglobulin based antibodies using non-immunoglobulin scaffolds onto which CDRs of the sEcad antibody can be grafted. Any non-immunoglobulin framework and scaffold know to those in the art may be used, as long as the framework or scaffold includes a binding region specific for the target. Immunoglobulin-like molecules include proteins that share certain structural features with immunoglobulins, for example, a β-sheet secondary structure. Examples of non-immunoglobulin frameworks or scaffolds include, but are not limited to, adnectins (fibronectin), ankyrin, domain antibodies and Ablynx nv, lipocalin, small modular immuno-pharmaceuticals (Trubion Pharmaceuticals Inc., Seattle, Wash.), maxybodies (Avidia, Inc., Mountain View, Calif.), Protein A and affilin (gamma-crystallin or ubiquitin) (Scil Proteins GmbH, Halle, Germany).

The anti-sEcad antibodies of the invention specifically bind to an epitope on the second, third, fourth or fifth subdomain of E-cadherin or sEcad (but not to an epitope of EC1). An epitope refers to an antigenic determinant on a target that is specifically bound by the paratope, i.e., the binding site of an antibody. Epitopic determinants usually consist of chemically active surface groupings of molecules such as amino acids or sugar side chains, and typically have specific three-dimensional structural characteristics, as well as specific charge characteristics. Epitopes generally have between about 4 to about 10 contiguous amino acids (a continuous epitope), or alternatively can be a set of noncontiguous amino acids that define a particular structure (e.g., a conformational epitope). Thus, an epitope can consist of at least 4, at least 6, at least 8, at least 10, or at least 12 such amino acids. Methods of determining the spatial conformation of amino acids are known in the art, and include, for example, x-ray crystallography and 2-dimensional nuclear magnetic resonance.

Methods of predicting other potential epitopes to which an antibody can bind are well-known to those of skill in the art and include without limitation, Kyte-Doolittle Analysis (Kyte and Dolittle, J. Mol. Biol. 157:105-132, 1982), Hopp and Woods Analysis (Hopp and Woods, Proc. Natl. Acad. Sci. USA 78:3824-3828, 1981; Hopp and Woods, Mol. Immunol. 20:483-489, 1983; Hopp, J. Immunol. Methods 88:1-18, 1986), Jameson-Wolf Analysis (Jameson and Wolf, Comput. Appl. Biosci. 4:181-186, 1988), and Emini Analysis (Emini et al., Virology 140:13-20, 1985). In some embodiments, potential epitopes are identified by determining theoretical extracellular domains. Analysis algorithms such as TMpred (see Hofmann and Stoffel, Biol. Chem. 374:166, 1993) or TMHMM (Krogh et al., J. Mol. Biol., 305(3):567-580, 2001) can be used to make such predictions. Other algorithms, such as SignalP 3.0 (Bednsten et al., J. Mol. Biol. 340(4):783-795, 2004) can be used to predict the presence of signal peptides and to predict where those peptides would be cleaved from the full-length protein. The portions of the proteins on the outside of the cell can serve as targets for antibody interaction.

The compositions of the present invention include antibodies that (1) exhibit a threshold level of binding activity; and/or (2) do not significantly cross-react with known related polypeptide molecules. The binding affinity of an antibody can be readily determined by one of ordinary skill in the art, for example, by Scatchard analysis (Scatchard, Ann. NY Acad, Sci. 51:660-672, 1949).

In some embodiments, the anti-sEcad antibodies can bind to their target epitopes or mimetic decoys at least 1.5-fold, 2-fold, 5-fold 10-fold, 100-fold, 10³-fold, 10⁴-fold, 10⁵-fold, 10⁶-fold or greater for the target second, third, fourth or fifth subdomain of sEcad than to other proteins predicted to have some homology to the second, third, fourth or fifth subdomain of E-cadherin or sEcad.

In some embodiments the anti-sEcad antibodies bind with high affinity of 10⁻⁴M or less, 10⁻⁷M or less, 10⁻⁹M or less or with subnanomolar affinity (0.9, 0.8, 0.7, 0.6, 0.5, 0.4, 0.3, 0.2, 0.1 nM or even less). In some embodiments the binding affinity of the antibodies for the second, third, fourth or fifth subdomain of sEcad is at least 1×10⁶ Ka. In some embodiments the binding affinity of the antibodies for the second, third, fourth or fifth subdomain of sEcad is at least 5×10⁶ Ka, at least 1×10⁷ Ka, at least 2×10⁷ Ka, at least 1×10⁸ Ka, or greater. Antibodies may also be described or specified in terms of their binding affinity to the second, third, fourth or fifth subdomain of sEcad. In some embodiments binding affinities include those with a Kd less than 5×10⁻² M, 10⁻² M, 5×10⁻³ M, 10⁻³ M, 5×10⁻³M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10-M, 5×10⁻⁹ M, 5×10⁻¹⁰ M, 10⁻¹⁰ M, 5×10⁻¹¹ M, 10⁻¹¹M, 5×10⁻¹²M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M, or less.

In some embodiments, the antibodies do not bind to known related polypeptide molecules; for example, they bind the second, third, fourth or fifth subdomain of a sEcad polypeptide but not known related polypeptides. Antibodies may be screened against known related polypeptides to isolate an antibody population that specifically binds to second, third, fourth or fifth subdomain of a sEcad polypeptide. For example, antibodies specific to second, third, fourth or fifth subdomain of a sEcad polypeptide will flow through a column comprising second, third, fourth or fifth subdomain of a sEcad polypeptide-related proteins (with the exception of second, third, fourth or fifth subdomain of a sEcad polypeptide) adhered to insoluble matrix under appropriate buffer conditions. Such screening allows isolation of polyclonal and monoclonal antibodies non-crossreactive to closely related polypeptides (Antibodies: A Laboratory Manual, Harlow and Lane (eds.), Cold Spring Harbor Laboratory Press, 1988; Current so Protocols in Immunology, Cooligan et al. (eds.), National Institutes of Health, John Wiley and Sons, Inc., 1995). Screening and isolation of specific antibodies is well known in the art (see, Fundamental Immunology, Paul (eds.), Raven Press, 1993; Getzoff et al., Adv. in Immunol. 43:1-98, 1988; Monoclonal Antibodies: Principles and Practice, Goding, J. W. (eds.), Academic Press Ltd., 1996; Benjamin et al., Ann. Rev. Immunol. 2:67-101, 1984). Representative examples of such assays include: concurrent immunoelectrophoresis, radioimmunoassay (RIA), radioimmunoprecipitation, enzyme-linked immunosorbent assay (ELISA), dot blot or Western blot assay, inhibition or competition assay, and sandwich assay.

The ability of a particular antibody to selectively kill malignant e-cadherin expressing cells can be evaluated using, for example, the methods disclosed in the Examples herein.

The anti-sEcad antibodies can include a tag, which may also be referred to as a reporter or marker (e.g., a detectable marker). A detectable marker can be any molecule that is covalently linked to anti-sEcad antibody or a biologically active fragment thereof that allows for qualitative and/or quantitative assessment of the expression or activity of the tagged peptide. The activity can include a biological activity, a physico-chemical activity, or a combination thereof. Both the form and position of the detectable marker can vary, as long as the labeled antibody retains biological activity. Many different markers can be used, and the choice of a particular marker will depend upon the desired application. Labeled anti-sEcad antibodies can be used, for example, for assessing the levels of sEcad in a biological sample, e.g., urine, salive, cerebrospinal fluid, blood or a biopsy sample or for evaluation the clinical response to sEcad peptide therapeutics. Suitable markers include, for example, enzymes, photo-affinity ligands, radioisotopes, and fluorescent or chemiluminescent compounds. Methods of introducing detectable markers into peptides are well known in the art. Markers can be added during synthesis or post-synthetically. Recombinant anti-sEcad antibodies or biologically active variants thereof can also be labeled by the addition of labeled precursors (e.g., radiolabeled amino acids) to the culture medium in which the transformed cells are grown. In some embodiments, analogues or variants of peptides can be used in order to facilitate incorporation of detectable markers. For example, any N-terminal phenylalanine residue can be replaced with a closely related aromatic amino acid, such as tyrosine, that can be easily labeled with ¹²⁵I. In some embodiments, additional functional groups that support effective labeling can be added to the fragments of an anti-sEcad antibody or biologically active variants thereof. For example, a 3-tributyltinbenzoyl group can be added to the N-terminus of the native structure; subsequent displacement of the tributyltin group with ¹²⁵I will generate a radiolabeled iodobenzoyl group.

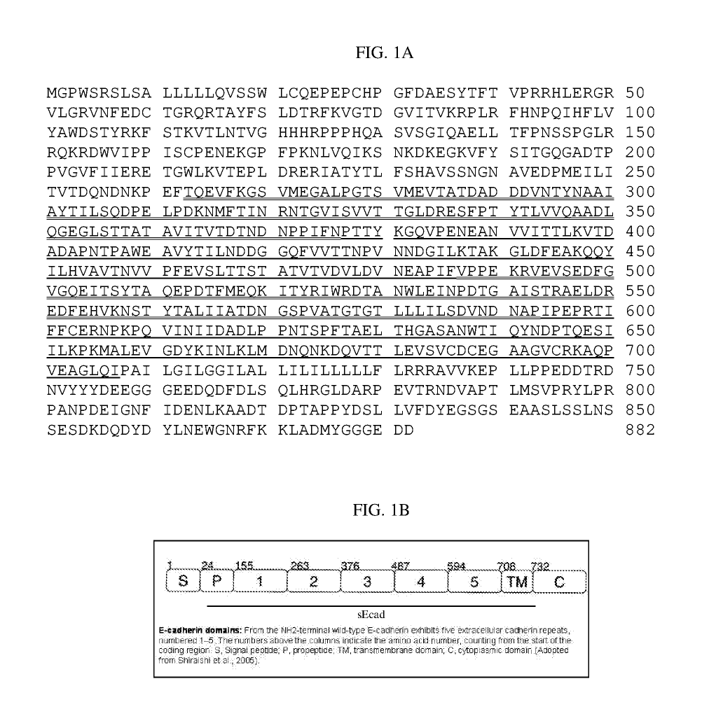

In lieu of administering an antibody or antibody-like therapeutic per se, the present methods can also be carried out by administering a protein that elicits the production of anti-sEcad antibodies in vivo. Accordingly, the compositions of the invention include antigenic fragments of the extracellular domain of E-cadherin in the EC2-EC5 subdomains (see FIG. 1A and FIG. 1B). These polypeptides can be fused to a heterologous polypeptide to generate an immunogenic fusion protein. For example, an sEcad polypeptide can be fused to a fragment of the influenza virus HA2 hemagglutinin protein as described in U.S. Pat. No. 7,262,270.

Also within the scope of the invention are nucleic acids that can be used to inhibit the expression of E-cadherin (e.g., an antisense oligonucleotide or an oligonucleotide that mediates RNA interference). Nucleic acid constructs can also be used to express antigenic fragments of sEcad in vivo or ex vivo (e.g., in cell or tissue culture).

The terms “nucleic acid” and “polynucleotide” may be used interchangeably herein to refer to agents useful in the context of the present methods, and these terms refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. In the context of the present invention, nucleic acids can encode, for example, an antibody, a mutant antibody or fragment thereof or a sEcad or fragment thereof.

An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among many (e.g., dozens, or hundreds to millions) of other nucleic acids within, for example, cDNA libraries or genomic libraries, or gel slices containing a genomic DNA restriction digest, is not an isolated nucleic acid.

Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein (i.e. an engineered protein). PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid (as one may wish to do, for example, when making an engineered protein, for example, an antibody, a mutant antibody or fragment thereof, or a fusion protein or fragment thereof. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, for example, a naturally occurring portion of an engineered protein-encoding DNA.

The nucleic acids and polypeptides described herein (e.g., antigenic fragments of sEcad) may be referred to as “exogenous”. The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

Recombinant constructs are also provided herein and can be used to transform cells in order to express a polypeptide, for example, an antibody, a mutant antibody or fragment thereof, or a sEcad or fragment thereof. A recombinant nucleic acid construct comprises a nucleic acid encoding, for example, an antibody, a mutant antibody or fragment thereof or a sEcad or fragment thereof as described herein, operably linked to a regulatory region suitable for expressing the engineered protein, for example, an antibody, a mutant antibody or fragment thereof or a sEcad or fragment thereof. In some cases, a recombinant nucleic acid construct can include a nucleic acid comprising a coding sequence, a gene, or a fragment of a coding sequence or gene in an antisense orientation so that the antisense strand of RNA is transcribed. It will be appreciated that a number of nucleic acids can encode a polypeptide having a particular amino acid sequence. The degeneracy of the genetic code is well known in the art. For many amino acids, there is more than one nucleotide triplet that serves as the codon for the amino acid. For example, codons in the coding sequence for a given fragment of an antibody, a mutant antibody or fragment thereof, or a fusion protein or fragment thereof can be modified such that optimal expression in a particular organism is obtained, using appropriate codon bias tables for that organism.

Vectors containing nucleic acids such as those described herein also are provided. A “vector” is a replicon, such as a plasmid, phage, or cosmid, into which another DNA segment may be inserted so as to bring about the replication of the inserted segment. Expression vectors include plasmid vectors, viral vectors, and the HSV amplicon particles as described in U.S. Application Publication No. 2006/0239970 (which is hereby incorporated herein by reference).

Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Also provided herein are host cells. A host cell can be for example, a prokaryote e.g., a bacterium such as E. coli, or a eukaryote, e.g., yeast, insect or mammalian cell that expresses a polypeptide of the present invention.

The agents described herein that inhibit sEcad can be included in pharmaceutical compositions that are physiologically acceptable (i.e., sufficiently non-toxic to be used in the therapeutic and prophylactic methods described herein). Accordingly, the invention features a variety of formulations, including topical creams (integrated into sunscreens) and sustained-release patches for transdermal delivery sEcad inhibitors. In other embodiments, the pharmaceutical composition can be formulated as an oral rinse, gel, or emulsion, or as a rectal solution, suspension, or emulsion. As will be apparent to one of ordinary skill in the art, the specific formulations can be selected based on the type of cancer being treated. For example, the oral rinse, gel, or emulsion can be used to treat cancers in the mouth, throat, esophagus, or stomach, and the rectal solution, suspension, or emulsion can be used to treat cancers in the rectum or colon. Formulations for oral and parenteral (e.g., intravenous) administration of a targeting agent are within the scope of the present invention.

The therapeutic agents of the invention (e.g., anti-sEcad antibodies and other protein- or nucleic acid-based agents that inhibit sEcad) can be formulated for administration to a patient with materials that improve their stability and/or provide for a controlled or sustained release in vivo. Accordingly, the invention encompasses delivery systems in which an sEcad-specific agent is formulated with microparticles (e.g., polymeric microparticles such as polylactide-co-glycolide microparticles) or nanoparticles (e.g., liposomes, polymeric carbohydrate nanoparticles, dendrimers, and carbon-based nanoparticles).

Other formulations include those for subcutaneous, intraperitoneal, intravenous, intraarterial, or pulmonary administration. As noted, sustained-release implants can also be made and used.

Any of the therapeutic or prophylactic methods of the invention can include a step of assessing a patient prior to treatment or as treatment progresses. As noted above, it is well documented that sEcad is elevated in the urine and/or serum of patients with breast, skin, lung, prostate, gastric and colorectal cancers as well as other epithelial malignancies. Consistent with earlier studies on sEcad levels, our data demonstrate that sEcad is shed at low levels from the surface of normal epithelial cells and at much higher levels from human skin cancer cells, human breast cancer cells, and mouse lung cancer cells. Thus, the present methods can include a step in which sEcad levels are determined from a sample (e.g., a urine or blood sample) obtained from a subject. Elevated levels are an indication that a subject is a good candidate for treatment as described herein, and monitoring sEcad as treatment progresses can help optimize dosing and scheduling as well as predict outcome. For example, monitoring can be used to detect the onset of resistance and to rapidly distinguish responsive patients from nonresponsive patients. Where there are signs of resistance or nonresponsivness, a physician can choose an alternative or adjunctive agent before the tumor develops additional escape mechanisms.

Compositions comprising two or more agents that specifically target one or more of the second, third, fourth or fifth subdomains of sEcad may be administered to persons or mammals suffering from, or predisposed to suffer from, cancer. The anti-sEcad antibodies may also be administered with another therapeutic agent, such as a cytotoxic agent, or cancer chemotherapeutic. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks.

The pharmaceutical compositions of the present invention can also include, in addition to an sEcad targeting agent, another therapeutic antibody (or antibodies (e.g., antibodies that recognize a cellular target (or targets) other than sEcad)). Exemplary immunoglobulins are listed below. Each immunoglobulin is identified by its proper name and its trade name. Numbers in parenthesis beginning with “DB” refer to the identifiers for each antibody on The DrugBank database available at the University of Alberta. The DrugBank database is described in Wishart et al., Nucl. Acids Res. 36:D901-906 (2008)) on the world wide web at www.drugbank.ca. Useful immunoglobulins include: Abciximab (ReoPro™) (DB00054), the Fab fragment of the chimeric human-murine monoclonal antibody 7E3, the synthesis of which is described in EP0418316 (A1) and WO 89/11538 (A1); Adalimumab (Humira™) (DB00051), a fully human monoclonal antibody that binds to Tumor Necrosis Factor alpha (TNF-alpha) and blocks TNF-alpha binding to its cognate receptor; alemtuzumab (Campath™) (DB00087), a humanized monoclonal antibody that targets CD52, a protein present on the surface of mature lymphocytes, used in the treatment of chronic lymphocytic leukemia (CLL), cutaneous T cell lymphoma (CTCL) and T-cell lymphoma; basiliximab (Simulect™) (DB00074), a chimeric mouse-human monoclonal antibody to the alpha chain (CD25) of the IL-2 receptor; bevacizumab (Avastin™) (DB00112) a humanized monoclonal antibody that recognises and blocks vascular endothelial growth factor (VEGF), the chemical signal that stimulates angiogenesis, the synthesis of which is described in Presta et al., Cancer Res., 57:4593-4599, 1997; certuximab (Erbitux™) (DB00002), a chimeric (mouse/human) monoclonal antibody that binds to and inhibits the epidermal growth factor receptor (EGFR), the synthesis of which is described in U.S. Pat. No. 6,217,866; certolizumab pegol (Cimzia™), a PEGylated Fab′ fragment of a humanized TNF inhibitor monoclonal antibody; daclizumab (Zenapax™) (DB00111), a humanized monoclonal antibody to the alpha subunit of the IL-2 receptor; eculizumab (Soliris™), a humanized monoclonal antibody that binds to the human C5 complement protein; efalizumab (Raptiva™) (DB00095), a humanized monoclonal antibody that binds to CD11a; gemtuzumab (Mylotarg™) (DB00056) a monoclonal antibody to CD33 linked to a cytotoxic agent, the amino acid sequence of which is described in J. Immunol. 148:1149 (1991), and Caron et al., Cancer. 73(3 Suppl):1049-1056, 1994); ibritumomab tiuxetan (Zevalin™) (DB00078), a monoclonal mouse IgG1 antibody ibritumomab in conjunction with the chelator tiuxetan and a radioactive isotope (yttrium⁹⁰ or indium¹¹¹); Infliximab (Remicade™) (DB00065), a chimeric mouse-human monoclonal antibody that binds to tumour necrosis factor alpha (TNF-alpha), the synthesis of which is described in U.S. Pat. No. 6,015,557; muromonab-CD3 (Orthoclone OKT3™), a mouse monoclonal IgG2a antibody that binds to the T cell receptor-CD3-complex; natalizumab (Tysabri™) (DB00108), a humanized monoclonal antibody against the cellular adhesion molecule α4-integrin, the sequence of which is described in Leger et al., Hum. Antibodies 8(1):3-16 (1997); omalizumab (Xolair™) (DB00043), a humanized IgG1k monoclonal antibody that selectively binds to human immunoglobulin E (IgE); palivizumab (Synagis™) (DB00110), a humanized monoclonal antibody (IgG) directed against an epitope in the A antigenic site of the F protein of the Respiratory Syncytial Virus (RSV), the amino acid sequence of which is described in Johnson et al., J. Infect. Dis. 176(5):1215-1224, 1997; panitumumab (Vectibix™), a fully human monoclonal antibody specific to the epidermal growth factor receptor (also known as EGF receptor, EGFR, ErbB-1 and HER1 in humans); ranibizumab (Lucentis™), an affinity matured anti-VEGF-A monoclonal antibody fragment derived from the same parent murine antibody as bevacizumab (Avastin™); rituximab (Rituxan™, Mabthera™) (DB00073), a chimeric monoclonal antibody against the protein CD20, which is primarily found on the surface of B cells; tositumomab (Bexxar™) (DB00081), an anti-CD20 mouse monoclonal antibody covalently bound to ¹³¹I; or trastuzumab (Herceptin™) (DB00072), a humanized monoclonal antibody that binds selectively to the HER2 protein.

The antibodies can include bioequivalents of the approved or marketed antibodies (biosimilars). A biosimilar can be for example, a presently known antibody having the same primary amino acid sequence as a marketed antibody, but one that may be made in a different cell type or by a different production, purification or formulation method than the marketed antibody. Generally, any deposited materials can be used.

The pharmaceutical compositions may also include or be administered along with a cytotoxic agent, e.g., a substance that inhibits or prevents the function of cells and/or causes destruction of cells. Exemplary cytotoxic agents include radioactive isotopes (e.g., ¹³¹I, ¹²⁵I, ⁹⁰Y and ¹⁸⁶Re), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are incorporated by reference herein). Such agents may be conjugated, coupled, linked or associated with an antibody or other targeting agent disclosed herein.

The pharmaceutical compositions may also include or be administered along with an agent that inhibits protein kinases. Kinases are enzymes that control the transfer of a phosphate group from a high energy phosphate donor such as adenosine-5′-triphosphate (ATP) to a specific substrate, such as a protein. The primary function of protein kinases is to turn on and off key cellular functions, including, but not limited to cell division. In some cases, protein kinases may remain in the “on” position indefinitely and lead to uncontrolled growth in a cell which may lead to the development of cancer. Examples of kinases that phosphorylate amino acids are tyrosine and serine/threonine. HER1, HER2 and HER3 are tyrosine kinase receptors of the EGFR family. Exemplary tyrosine kinases include agents that inhibit EGFR such as gefitinib (Iressa) and erlotinib (Tarceva); or the IFG-1 receptor such as AG1024 and NVP-ADW742; dual tyrosine kinase inhibitors that block both HER1 and HER2 pathways such as lapatinib (Tykerb) and HER dimerization inhibitors such as pertuzumab (Perjeta) that inhibits the dimerization of HER2 with other HER receptors. Exemplary serine/threonine protein kinases that inhibit mTOR include temsirolimus (Torisel); everolimus (Zortress; Afinitor), sirolimus (also known as rapamycin; Rapamune) and AZD8055; or p70S6K, which acts downstream of PIP3 in the P13 kinase pathway, include LY2584702. Examples of non-specific kinase inhibitors that may act on several receptors such as EGFR, VEGFR and RET-tyrosine kinase include vandetanib (Caprelsa) or block multiple members of the HER family (sometimes referred to as “pan-HER” inhibitors), include canertinib (Cl-1033, PD183805). Exemplary protein kinases that inhibit MEK include GDC-973 or PI3K include GDC-041, GSK105615 (Pan-PI3K), BKM120 and perifosine (KRX-0401). Exemplary inhibitors of Akt (a serine/threonine-specific protein kinase) include perifosine (KRX-0401) and MK2206. Any of the above kinase inhibitors may be administered in combination with another kinase inhibitor. Some of the agents above may have multiple mechanisms of therapeutic action and/or inhibit more than one step in a particular intracellular pathway. Thus, pharmaceutical compositions may also include or be administered along with an agent that inhibits both P13K and mTOR such as NVP-BEZ235 or P70S6K and Akt such as LY2780301.

Conventional cancer medicaments can be administered with the compositions disclosed herein. Useful medicaments include anti-angiogenic agents, i.e., agents block the ability of tumors to stimulate new blood vessel growth necessary for their survival. Any anti-angiogenic agent known to those in the art can be used, including agents such as Bevacizumab (Avastin®, Genentech, Inc.) and aflibercept (Zaltrap) that block the function of vascular endothelial growth factor (VEGF). Other examples include, without limitation, Dalteparin (Fragmin®), Suramin ABT-510, Combretastatin A4 Phosphate, Lenalidomide, LY317615 (Enzastaurin), Soy Isoflavone (Genistein; Soy Protein Isolate) AMG-706, Anti-VEGF antibody, AZD2171, Bay 43-9006 (Sorafenib tosylate), PI-88, PTK787/ZK 222584 (Vatalanib), SU11248 (Sunitinib malate), VEGF-Trap, XL184, ZD6474, Thalidomide, ATN-161, EMD 121974 (Cilenigtide) and Celecoxib (Celebrex®).

Other useful therapeutics include those agents that promote DNA-damage, e.g., double stranded breaks in cellular DNA, in cancer cells. Any form of DNA-damaging agent know to those of skill in the art can be used. DNA damage can typically be produced by radiation therapy and/or chemotherapy. Examples of radiation therapy include, without limitation, external radiation therapy and internal radiation therapy (also called brachytherapy). Energy sources for external radiation therapy include x-rays, gamma rays and particle beams; energy sources used in internal radiation include radioactive iodine (iodine¹²⁵ or iodine¹³¹), and from strontium⁸⁹, or radioisotopes of phosphorous, palladium, cesium, iridium, phosphate, or cobalt. Methods of administering radiation therapy are well known to those of ordinary skill in the art.

Examples of DNA-damaging chemotherapeutic agents include, without limitation, Busulfan (Myleran), Carboplatin (Paraplatin), Carmustine (BCNU), Chlorambucil (Leukeran), Cisplatin (Platinol), Cyclophosphamide (Cytoxan, Neosar), Dacarbazine (DTIC-Dome), Ifosfamide (Ifex), Lomustine (CCNU), Mechlorethamine (nitrogen mustard, Mustargen), Melphalan (Alkeran), and Procarbazine (Matulane).

Other standard cancer chemotherapeutic agents include, without limitation, alkylating agents, such as carboplatin and cisplatin; nitrogen mustard alkylating agents; nitrosourea alkylating agents, such as carmustine (BCNU); antimetabolites, such as methotrexate; folinic acid; purine analog antimetabolites, mercaptopurine; pyrimidine analog antimetabolites, such as fluorouracil (5-FU) and gemcitabine (Gemzar®); hormonal antineoplastics, such as goserelin, leuprolide, and tamoxifen; natural antineoplastics, such as aldesleukin, interleukin-2, docetaxel, etoposide (VP-16), interferon alfa, paclitaxel (Taxol®), and tretinoin (ATRA); antibiotic natural antineoplastics, such as bleomycin, dactinomycin, daunorubicin, doxorubicin, daunomycin and mitomycins including mitomycin C; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxin®), Schizophyllan, cytarabine (cytosine arabinoside), dacarbazine, thioinosine, thiotepa, tegafur, dolastatins, dolastatin analogs such as auristatin, CPT-11 (irinotecan), mitozantrone, vinorelbine, teniposide, aminopterin, carminomycin, esperamicins (See, e.g., U.S. Pat. No. 4,675,187), neocarzinostatin, OK-432, bleomycin, furtulon, broxuridine, busulfan, honvan, peplomycin, bestatin (Ubenimex®), interferon-β, mepitiostane, mitobronitol, melphalan, laminin peptides, lentinan, Coriolus versicolor extract, tegafur/uracil, estramustine (estrogen/mechlorethamine).

Additional agents which may be used as therapy for cancer patients include EPO, G-CSF, ganciclovir; antibiotics, leuprolide; meperidine; zidovudine (AZT); interleukins 1 through 18, including mutants and analogues; interferons or cytokines, such as interferons α, β, and γ hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; and anti-angiogenesis factors.

Useful therapeutic agents include, produgs, e.g., precursors or derivative forms of a pharmaceutically active substance that is less cytotoxic or non-cytotoxic to tumor cells compared to the parent drug and is capable of being enzymatically activated or converted into an active or the more active parent form. See, e.g., Wilman, Biochemical Society Transactions, 14:375-382 (1986) and Stella et al., “Prodrugs: A Chemical Approach to Targeted Drug Delivery,” Directed Drug Delivery, Borchardt et al., (ed.), pp. 247-267, Humana Press (1985). Prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, b-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use herein include, but are not limited to, those chemotherapeutic agents described above.

Any method known to those in the art can be used to determine if a particular response is induced. Clinical methods that assess the degree of a particular disease state can be used to determine if a response is induced. The particular methods used to evaluate a response will depend on the nature of the patient's disorder, the patient's age and sex, other drugs being administered and the judgment of the attending clinician.

### EXAMPLES

Although antibody-based therapy targeting the ErbB family of receptors (i.e. HER1-4) has dramatically transformed the treatment of metastatic breast and other epithelial-derived cancers, cardiac toxicity and severe anaphylaxis can occur. More importantly, patients on HER-MEK or mTOR targeted therapies eventually develop resistance as a result of compensatory upregulation of: 1) alternate HER family members; 2) other receptor tyrosine kinases (i.e. IGF-1R); 3) hyperactivation of downstream MAPK-PI3K/Akt/mTOR; and 4) activation of the inhibitor of apoptosis proteins (IAPs). Recent reports indicate that sEcad can act as a ligand for the HER2 receptor, whereby it induces the heterodimerization of HER2-HER3 and activation of Akt/Erk pathways in SKBR3 breast cancer cells. Here, we show that antibody-based therapy targeting specific domains of the extracellular domain of E-cadherin (i.e. EC2-5), including the shed sEcad fragment (EC2-5) (herein referred to as sEcad), inhibited proliferation and induced programmed cell death in human breast (HER2-positive, HER2-negative and HER2+Herceptin-resistant), colon, lung, prostate and mouse skin squamous cell cell lines. In breast (HER2-positive, HER2-negative and HER2-positive Herceptin resistant) and skin cancer cell lines, this anti-sEcad antibody therapy resulted in the down-regulation of multiple HER family members (i.e HER1-4), insulin-like growth factor 1 receptor (IGF-1R) and downstream PI3K/Akt/mTOR-IAP signaling. Importantly, in various preclinical breast and skin cancer mouse models in vivo, anti-sEcad mAb therapy significantly delayed tumor onset, attenuated tumor burden and decreased tumor grade by reducing proliferation and activating tumor cell apoptosis. Consistent with the cell culture studies, resected tumors from the antibody treated mice exhibited a significant reduction in HER1-4, MAPK-PI3K/Akt/mTOR and IAP expression levels. Taken together, our data show a novel micro-environmental tumor target that, depending on the cancer cell-type, down-regulates multiple receptor tyrosine kinases (HER1, HER2, HER3, HER4, IGF-1R) and PI3K/Akt/mTOR-IAP signaling, while sparing toxicity to mice, normal tissues and cells. In conclusion, the proposed treatment offers a novel therapeutic platform for breast or other epithelial cancers that may be used alone or in combination with other HER-MAPK-PI3K/Akt/mTOR-IAP targeted therapies to combat primary and acquired resistance.

### Example 1: In Vivo and In Vitro Efficacy of a Monoclonal Antibody Targeting sEcad

We tested the in vivo efficacy of a monoclonal antibody targeting sEcad using MMTV-PyMT transgenic mice in which mammary targeted transgenic expression of the polyomavirus middle T antigen (PyMT) leads to the rapid development of palpable tumors that progress to aggressive adenocarcinomas with metastasis to the lungs (Guy et al., Mol. Cell Biol. 12:954-961, 1992). Mice were treated weekly with saline, IgG or DECMA-1 (1 mg/kg in 200 μL saline; Sigma) beginning at 47 days of age until the mice were sacrificed at 90 days of age. Treatment with the anti-sEcad mAb resulted in a significant delay in tumor onset and decreased tumor burden (**p<0.01, ***p<0.001). Treated tumors exhibited reduced proliferation and enhanced apoptosis, the latter of which was associated with enhanced p53 expression. Similarly, in HER2-positive (HER2+) Herceptin-resistant breast cancer xenografts, anti-sEcad therapy resulted in a statistically significant reduction in tumor growth. Lastly, we replicated these findings in an orthotopic skin squamous cell carcinoma xenograft model, in which anti-sEcad administration resulted in a statistically significant reduction in tumor growth and tumor grade. As in the MMTV-PyMT model, resected skin tumors exhibited a marked reduction in proliferation and enhanced apoptosis, the latter of which was associated with increased p53 expression levels.

Next, we validated these findings in vitro, where anti-sEcad mAb treatment induced a significant dose-dependent increase in apoptosis in human breast (HER2+, HER2-, Herceptin resistant), lung, colon, head and neck, prostate and skin SCC cell lines, and in those tested, this apoptosis was associated with enhanced p53 and Bad levels. In addition to eliciting cell death, anti-sEcad mAb treatment significantly decreased the proportion of BrdU-positive proliferating cells, consistent with the observations in our preclinical mouse studies. Because the EC1-2 domains and not the EC3-5 domains are indispensable for homophilic adhesion (Shiraishi et al., J. Immunol. 175:1014-1021, 2005), this prompted us to examine whether anti-sEcad mAb targeted therapy (EC3-5) exhibited any cytotoxicity in cell culture systems or in vivo. Confluent human mammary epithelial cells (MCF-10A), primary human keratinocytes, melanocytes and fibroblasts exhibited no statistically significant induction of apoptosis following anti-sEcad mAb treatment compared to isotype IgG controls, and no apparent toxicity was observed in excised heart, liver, intestine and kidneys in anti-sEcad-treated MMTV-PyMT mice or SCC xenografts as evaluated by gross exam and pathologic analysis of hematoxylin and eosin-stained paraffin-embedded sections. Collectively, these data show that targeted mAb therapy against specific ectodomains of E-cadherin (EC2-5), including ectodomains of the shed sEcad fragment, inhibits cancer growth and induces tumor cell apoptosis without producing untoward toxic effects on non-tumorigenic cells, mice or end-organs.

The methods employed in the study described above included the following:

Cell Lines and Reagents.

Human MCF-7, NCI-H292, BT474 parental, PC3, HT29, FADU, Detroit, MCF-10A, WI-38, primary epidermal keratinocytes (PHK), and primary epidermal melanocytes were purchased from the American Type Culture Collection (ATCC), and cultured according to ATCC recommendations. BT474-Herceptin resistant cells were a kind gift of Dr. Kute (Wake Forest University, Winston-Salem, N.C.) and grown in 10% FBS RPMI-1640 medium (ATCC). PAM212 cells were a kind gift from Dr. Yuspa (NCI, Bethesda, Md.) and grown in Dulbecco's modified Eagle's medium (DMEM; Lonza) with 10% FBS. Anti-sEcad ectodomain-specific antibodies include DECMA-1 (Sigma), HECD-1 (Calbiochem), SHE78-7 (Calbiochem) and H108 (Santa Cruz Biotechnology).

In Vivo MMTV-PyMT Studies.

MMT-PyMT mice were purchased from the Jackson Laboratory and maintained the colony by breeding hemizygous males with FVB wild-type females. When mice reached 47 days of age, female mice were randomly assigned to groups (n=5, each group) to receive weekly intraperitoneal injections of 1 mg/kg DECMA-1, rat IgG1, or equivalent volumes of saline. Palpable tumors were monitored twice weekly until sacrifice at 90 days of age. Breast tumors were excised, weighed and either formalin fixed or flash frozen. Formalin fixed samples were paraffin embedded, cut at 5 μm and either H&E stained for histological evaluation or processed for immunohistochemical analysis.

In Vivo Tumor Xenograft Studies.

Female severe combined immunodeficient mice (SCID) or athymic nude mice (nu/nu) mice were obtained from Taconic. 6-8 week old mice were injected subcutaneously (SQ) with exponentially growing 1×10⁶ PAM212 or 1×10⁶ BT474Herceptin resistant cells or 1×10⁶ MDA-MB-231 cells in 200 μl of saline. For the PAM212 xenografts, mice were randomized into receiving a single intraperitoneal dose of saline (n=6), IgG (10 mg/kg; n=6) or anti-sEcad mAb (DECMA-1, 10 mg/kg, n=7) when tumors reached 100 mm³. Treatments were continued for 20 days. For B474Herceptin resistant xenografts (preliminary studies), mice were randomized to receive one dose of saline, IgG (20 mg/kg) or anti-sEcad mAb (DECMA-1, 20 mg/kg) once tumors reached 100 mm³ and sacrificed 2 weeks after treatment. Mice were then observed twice weekly and the growth of each lesion was recorded and digital photographs at a fixed distance taken. Tumor volume (mm³) was assessed using Vernier calipers and calculated as l×w²×0.52, where l represents length and w width (Euhus et al., J. Surg. Oncol. 31; 229-234, 1986). Skin and breast tumors were excised, weighed and either formalin fixed or flash frozen. Formalin fixed samples were paraffin embedded, cut at 5 μm and either H&E stained for histological evaluation or processed for immunohistochemical analysis.

Assessment of Apoptosis, Cell Number and Proliferation.

Cellular apoptosis was quantitatively determined using the Cell Death Apoptosis Detection ELISA Plus kit (Boehringer Mannheim), which is based on the detection of DNA-histone complexes in the form of mono- and oligo-nucleosomes, according to the manufacturer's instructions. Briefly, cells were treated in the presence or absence of various concentrations of DECMA-1 or rat IgG1 for 24 to 48 h. Cells were harvested by trypsinization, counted and lysates were subjected to the ELISA. In vitro and in vivo apoptosis was further analyzed by in situ detection of fragmented DNA, using the DeadEnd Fluorometric TUNEL System (Promega), the ApopTag Peroxidase In situ Apoptosis Detection Kit (Millipore) and the human Apoptosis Array Kit (ARY009, R & D), according to the manufacturer's instructions. The ApopTag kit was performed on deparaffinized 5 μm-thick sections treated with proteinase K (20 μg/ml) at RT. For cell counting, the cells were trypsinized and counted on a haemocytometer. For tumor BrdU analyses, tissues were stained using an anti-BrdU (ab2284, abcam) antibody, as previously described (27). The proliferation of cells was monitored by a cell proliferation ELISA 5-bromo-2′-deoxyuridine (BrdU) (colorimetric) kit (Roche, Stockholm, Sweden), according to the manufacturers protocol.

Statistical Analysis.

Comparisons between groups were made using ANOVA followed by post-hoc analysis using the Student-Newman-Keuls or Dunnett's method unless otherwise stated. Statistical significance is indicated in figures as *P<0.05, **P<0.01 or ***P<0.001

### Example 2: Monoclonal Antibody Against sEcad Inhibits Carcinogenesis by Downregulating Multiple Receptor Tyrosine Kinases and Downstream MAPK-PI3K/Akt/mTOR and IAP Pathways

Since the HER family of receptors are integral to the growth and progression of cancers, we next evaluated whether our anti-sEcad therapy suppressed tumorigenesis by modulating this pro-oncogenic pathway. Our studies show that in the treated MMTV-PyMT (primarily HER1 and HER2) and skin SCC xenograft tumors (primarily HER1, IGF-1R) there was a statistically significant decrease in HER1, HER2 and IGF-1R expression levels. As clinical trials combining HER targeted agents with inhibitors of the PI3K/mTOR pathway are showing promise in patients (Hennessy et al. Nat. Rev. 4:988-1004, 2005), we next examined MAPK-PI3K/Akt/mTOR and IAP (i.e. survivin, livin, XIAP, c-IAP-1, c-IAP2 etc.) expression levels in the resected tumors from MMTV-PyMT and skin SCC xenografts. Treated MMTV-PyMT tumors exhibited marked decreases in MEK1/2, ERK1/2, PI3K, Akt, mTOR, 4EBP1 (mTOR substrate) and survivin levels compared to saline or IgG controls. Similarly, a marked decrease in HER and IGF-1R levels of expression, as well as Ras, MEK1/2 and 4EBP-1 levels were evident in resected anti-sEcad SCC tumors (data still in progress).

To validate these in vivo findings, we next examined how anti-sEcad monoclonal antibody (mAb) administration affected these receptor tyrosine kinases and downstream MAPK-PI3K/Akt/mTOR-IAP signaling in cell culture systems. Western blot analyses of membrane, cytosolic and nuclear fractions of HER2+ MCF-7 cells revealed a marked anti-sEcad mAb-mediated reduction in HER1-4 levels of expression, as well as reductions in the expression levels of MAPK-PI3K/Akt/mTOR and the IAPs survivin, XIAP and c-IAP-1. Similarly, in BT474-Herceptin resistant cells, anti-sEcad mAb treatment resulted in the downregulation of phospho-HER1, HER2, IGF-1R, as well as MEK, ERK1/2, PI3K, Akt, mTOR, 4EBP1 and the IAPs survivin, livin and XIAP. In skin SCC cells, a similar anti-sEcad-induced reduction in HER1-2 (don't express HER3, 4), IGF-1R and downstream MEK, ERK, PI3K, mTOR, 4EBP1 as well as survivin levels was observed. Taken together, blockade of sEcad resulted in the down-regulation of multiple receptor tyrosine kinases (HER1-4, IGF-1R etc) as well as downstream PI3K/mTOR signaling in Herceptin-sensitive and resistant cell lines, suggesting that this therapy may be efficacious in those patients with de novo and acquired resistance to HER targeted therapies.

Endocytic trafficking and degradation of the HER family of receptors provides an important mechanism of action of multiple HER-directed cancer therapies (Hennessy et al., supra; Sorkin and Goh, Exp. Cell Res. 314:3093-3106, 2008). Therefore, we next determined whether mAb targeting sEcad induced the internalization and degradation of HER receptors. Immunofluorescence staining of HER1-4 in untreated MCF-7 cells demonstrated a predominant plasma membrane localization, but anti-sEcad mAb treatment induced the aggregation of HER1-4 immunoreactivity into intracellular vesicular structures. Subsequent experiments suggested that anti-sEcad mAb treatment induces tumor cell apoptosis via directing the HER receptor family for endocytosis and degradation. Chloroquine, a lysosomal inhibitor, prevented anti-sEcad mAb-induced apoptosis at low doses and rescued HER1-4 membrane expression. In addition, the effect of the anti-sEcad mAb on apoptosis was inhibited in the presence of the proteasome inhibitor ALLN and accompanied by the rescue of HER1-4 expression. Indeed, the ubiquitination of HER1 and HER2 was dramatically increased in the cytosolic and membrane fractions of anti-sEcad mAb treated cells. This data suggests that the lysosome and ubiquitin-proteasome pathways mediate the effects of anti-sEcad mAb therapy on HER receptor expression and apoptosis in cancer cells.

In conclusion, our findings demonstrate that the administration of antibodies against specific ectodomains of E-cadherin (EC2-5), including the shed sEcad fragment (EC2-5), significantly decreased tumor burden and reduced tumor grade in the MMTV-PyMT and SCC xenograft mouse models of cancer by retarding cancer cell proliferation and inducing apoptosis selectively in cancer cells without untoward cytotoxicity in normal cells, mice and end-organs. Mechanism-of-action studies reveal a previously undiscovered mechanism, whereby anti-sEcad monoclonal antibody therapy down-regulated the HER family receptors via endocytosis and degradation by the ubiquitin-proteasome and lysosomal pathways and also down-regulated distal PI3K/Akt/mTOR-IAP signaling, therefore potentially overcoming the development of host resistance often seen in response to existing therapies. Therefore, anti-sEcad targeted monoclonal antibody therapy may be proven as an effective therapy, administered alone or in combination with other strategies, for the treatment of epithelial-derived cancers. To this end, our results clearly demonstrate that anti-sEcad antibody treatment induces cell death in a variety of epithelial cancer cell lines (breast, skin, colon, Head & Neck, lung etc.), while sparing adjacent normal healthy epithelial cells, fibroblasts and endothelial cells. We propose that cancer cells secrete sEcad in the microenvironment to artificially mimic normal cell-cell contacts, promote sEcad-receptor tyrosine kinase (RTK) interactions and by providing a functional scaffolding with adjacent neighboring cells. Thus, by scavenging sEcad from the tumor microenvironment and inhibiting sEcad-RTK interactions, we perturb this nurturing capacity of the tumor cell mileu and activate p53-dependent or independent molecular pathways involved in programmed cell death.

The methods employed in the study described above included the following:

Cell Lines and Reagents.

Human MCF-7 and BT474 parental cells were purchased from the American Type Culture Collection (ATCC), and cultured according to ATCC recommendations. BT474-Herceptin resistant cells were a kind gift of Dr. Kute (Wake Forest University, Winston-Salem, N.C.) and grown in 10% FBS RPMI-1640 medium (ATCC). Anti-sEcad ectodomain-specific antibodies include DECMA-1 (Sigma), HECD-1 (Calbiochem), SHE78-7 (Calbiochem) and H108 (Santa Cruz Biotechnology). The IAP antibody sampler kit was purchased from Cell Signaling. ALLN proteasome and chloroquine lysosome inhibitors were obtained from Sigma.

Fractionation, Immunoprecipitation and Western Blot Analyses.

Subcellular fractionation was performed using the BioVision FractionPREP Cell Fractionation System (BioVision Inc. Mountain View, Calif.), as per the manufacturer's instructions. We carried out immunoprecipitation assays by harvesting cells, in the presence or absence of DECMA-1 or control rat IgG1, with immunoprecipitation lysis buffer (20 mM Tris-HCl, pH 7.5; 137 mM NaCl; 100 mM NaF; 10% glycerol (vol/vol); 1.0% (vol/vol) Nonidet P-40; 1 mM PMSF and protease inhibitor cocktail (Sigma). After brief sonication, the lysates were cleared by centrifugation at 4° C. Supernatants were incubated with EGFR/HER1 (Ab-15) and HER2 (Ab-17)-specific antibodies from Thermo Fisher Scientific for 4 hours and protein A/G plus agarose beads (Santa Cruz, sc-2003) for 2 hours at 4° C. The immunocomplexes were washed three times, boiled in sample buffer (60 mM Tris-Cl, pH 6.8; 2% SDS (vol/vol); 10% glycerol (vol/vol); 5% 3-mercaptoethanol (vol/vol); and 0.01% bromophenol blue (vol/vol), and loaded on SDS-PAGE for protein analysis. Whole-cell extracts from tumors or tumor cells were processed for western blots as described previously (Brouxhon et al., Cancer Res. 67:7654-64, 2007). Antibodies against total HER1, HER2, HER3 and HER4 and phosphorylated HER1-4, pPI3K, pAkt, pmTOR, p4E-BP1, pp70S6K and survivin (2808) were obtained from Cell Signaling Technologies. Monoclonal antibodies to HER2, HER3 and HER4 were obtained from Thermo Scientific. Polyclonal antibody to the extracellular domain of E-cadherin and monoclonal antibody to α-actin were obtained from Santa Cruz Biotechnology. Monoclonal antibodies to the extracellular domain of E-cadherin (DECMA-1) and to His Tag were obtained from Sigma and Abcam, respectively. Monoclonal antibodies to G3PDH, ubiquitin and rat IgG1 were obtained from Ambion, Zymed and Southern Biotechnology, respectively. The human Apoptosis Array Kit was purchased from R&D Systems (ARY009) and used according to the manufacturer's instructions.

Immunofluorescence.

Cells cultured on chamber slides (Nalge Nunc International) were fixed with 4% formaldehyde for 10 minutes and blocked for 20 minutes in PBS containing 1% (wt/vol) BSA and incubated in HER1-4 antibodies, as previously described (Brouxhon et al., Cancer Res. 67:7654-64, 2007). Nuclei were counterstained with 2 μg/ml Hoechst 33342 (Invitrogen).

Histological Analysis and Immunohistochemistry.

Paraffin-embedded tumors were fixed, sectioned at 5 μm, deparaffinized and stained with H&E according to standard protocols. H&E-stained slides were blindly evaluated by board-certified pathologists. For immunohistochemical analyses, tissues were stained using anti-HER1, HER2, HER3 HER4, p53, ERK1/2, Akt, mTOR and survivin (Cell Signaling) antibodies, as previously described (Brouxhon et al., Cancer Res. 67:7654-64, 2007).

Targeted monoclonal antibodies have become attractive therapeutic drug candidates due to their potential for tumor-specific targeting and low toxicity profiles. Accordingly, Trastuzumab, a recombinant humanized monoclonal antibody against the extracellular domain of HER2, was approved by the FDA for the treatment of patients with HER2 metastatic breast cancers (Yarden and Sliwkowski, Nat Rev Mol Cell Biol. 2:127-37, 2001). However, a large percentage of patients who are initially responsive to HER-targeted therapies experience tumor recurrence and become refractory to therapy (Yarden and Sliwkowski, Nat Rev Mol Cell Biol. 2:127-37, 2001). Extensive crosstalk between multiple HER receptors and downstream key survival-signaling pathways, have been suggested to contribute to this drug resistance (Nahta et al., Nat Clin Pract Oncol. 3:269-80, 2003). Accordingly, bi-specific antibodies that simultaneously neutralize HER2 and HER3, or combinations of targeted mAb that inhibit HER1 and HER2 alone or target these receptor tyrosine kinases in combination with downstream MAPK or PI3K/Akt/mTOR inhibitors are currently being actively pursued in clinical trials (Robinson et al., Br J Cancer. 99:1415-25, 2008).

To gain a better understanding of the putative mechanisms by which anti-sEcad administration suppressed the development of carcinomas, we next examined whether anti-sEcad therapy modulated the HER family of receptors. Importantly, the most intriguing and clinically relevant finding of this study is the demonstration that anti-sEcad therapy down-regulated many of the compensatory pathways that Trastuzumab-treated cells eventually develop so as to sustain cell proliferation and enhance survival. That is, one of the most important strategies to improve the efficacy of HER-targeted therapies is to inhibit multiple HER receptors, thereby interfering with the horizontal cooperatively that exists between these receptors that currently limit the success of agents that target individual receptors. Specifically, simultaneous use of trastuzumab, pertuzumab and the HER tyrosine kinase inhibitor gefitinib inhibited HER2-overexpressing xenografts more effectively than any of these drugs used as a single agent or in dual combinations (Serra et al., Oncogene. 30:2547-57, 2011). Here, our mechanistic studies reveal a previously undiscovered pathway, wherein anti-sEcad treatment down-regulated all HER1-4 family members, with the HER1 and HER2 family members being endocytosed and degraded by the ubiquitin-proteasome and lysosome pathways. However, downregulation of all HER family members may not be sufficient, since acquired drug resistance can still occur through reactivation of downstream pro-survival signaling pathways.

The PI3K/Akt/mTOR and MAPK pathways, along with IAP family members, are highly dysregulated in breast cancer (Nahta et al., Nat Clin Pract Oncol. 3:269-80, 2003), making them well-validated targets for cancer treatment. However, with extensive cross-talk and many feedback loops, results of single agent inhibitors have only had modest effects. This was demonstrated by Serra et al (2011) who showed that sole PI3K targeting in HER2-overexpressing breast cancers activated alternate compensatory pathways resulting in ERK dependency (Serra et al., Oncogene. 30:2547-57, 2011). Moreover, Carracedo et al (2008), demonstrated that inhibition of mTORC1, using rapamycin, resulted in MAPK activation through a PI3K feedback loop in prostate cancer (Carracedo et al., J Clin Invest. 118:3065-74, 2008). Here, we show that anti-sEcad mAb treatment suppressed many of the complex and redundant downstream pathways involved in Trastuzumab resistance. Specifically, membrane fractionation of mAb treated MCF-7 cells resulted in the downregulation of ERK1/2 as well as Akt, mTOR, the mTOR-binding protein Raptor and the mTOR substrate 4E-BP1. Consistent with the in vitro findings, PI3K/Akt/mTOR signaling was also significantly reduced in tumors from mAb treated MMTV-PyMT mice. Recent studies also demonstrate that IAPs are indispensable for survival of HER2-positive breast cancer cells. Xia et al (2006) showed that the HER1/HER2 inhibitor, lapatinib, markedly reduced survivin expression and induced apoptosis through its inhibition of PI3K signaling in HER2 overexpressing BT474 cells (Xia et al., Proc Natl Acad Sci USA. 103:7795-800, 2006). Similarly, HER2-positive tumors from patients treated with lapatinib exhibited a marked inhibition of survivin expression levels (Asanuma et al., Cancer Res. 65:11018-25, 2005; Xia et al., Proc Natl Acad Sci USA. 103:7795-800, 2006). In contrast, Trastuzumab had little effect on steady-state survivin levels in Trastuzumab-sensitive BT474 cells, but in the HER2-positive Trastuzumab-resistant SUM190PT cell line, it induced the upregulation of both survivin and XIAP (Aird et al., Mol Cancer Ther. 7:38-47, 2008; Xia et al., Proc Natl Acad Sci USA. 103:7795-800, 2006). In this study, we further show that anti-sEcad therapy down-regulated the inhibitor of apoptosis protein family members survivin, XIAP, livin and c-IAP-1, many of which are essential for tumor cell survival and are also upregulated in resistant tumors (Xia et al., Proc Natl Acad Sci USA 103:7795-800, 2006).

### Example 3 sEcad is Increased in Human and Mouse Cancers and Cell Culture Systems

As sEcad upregulation has been previously reported in the serum of cancer patients (Katayama et al., Br J Cancer. 69: 580-585, 1994), we first set out to determine whether endogenous sEcad levels are increased in human or mouse cancer specimens, bodily fluids or cell culture systems. To this end, we first assessed the level of sEcad expression in HER2+ human breast tumor specimens and human triple negative breast cancer (TNBC) tumor specimens and found sEcad levels to be significantly higher than normal human mammary tissue specimens. In human cell culture studies, sEcad levels were found to be significantly increased in the conditioned media of MCF-7 breast cancer cells compared to normal MCF-10A breast epithelial cells. In contrast, no appreciable level of sEcad was found in HER2+ SKBR3 and TNBC MDA-MB-231 cells, which can be attributed to the lack of E-cadherin expression in these cell lines. In the MMTV-PyMT mice bearing HER2+ breast tumors, increased amounts of endogenous sEcad levels were exhibited in resected breast tumors, as well as heightened levels of sEcad in the serum and urine compared to wild-type controls.

In human skin and head & neck specimens, immunoblot analysis showed a statistically significant decrease in intact E-cadherin (FL-Ecad) and a parallel increase of the 80 kDa sEcad fragment in skin and head & neck squamous cell carcinomas (SCCs) versus normal skin and oropharyngeal epidermal samples (Brouxhon et al., Oncogene 2012; In press). Furthermore, human skin and head & neck SCC cell lines demonstrated a statistically significant increase in sEcad shedding versus controls, as assessed by ELISA and immunoblot analyses. This inverse FL-Ecad/sEcad relationship was also confirmed in a chronic photocarcinogenesis skin cancer mouse model comprised of areas of dysplasia, resected papillomas and SCCs (Brouxhon et al., Oncogene 2012; In press). Interestingly, FL-Ecad mRNA levels increased with UV-induced skin cancer progression. Using an in vitro SCC skin cancer progression model, we further validated that sEcad secreted levels increased in the conditioned media of cells with progression from normal skin keratinocytes to papillomas and SCCs (Brouxhon et al., Oncogene 2012; In press). Taken together, our data demonstrate that endogenous sEcad levels are increased in an array of human and mouse cancer specimens, cell lines and bodily fluids.

The methods employed in the study described above included the following:

Cell Lines and Reagents.

Human MCF-7, MCF-10A, SKBR3, MD-MB-231 and FADU cells were purchased from ATCC, and cultured according to ATCC recommendations. PAM212 cells were a kind gift from Dr. Yuspa (NCI, Bethesda, Md.) and grown in Dulbecco's modified Eagle's medium (DMEM; Lonza) with 10% FBS. CC4A cells were kindly provided by Dr. Klein-Szanto (Fox Chase Cancer Center, Philadelphia, Pa.) and cultured in SMEM medium supplemented with 10% FBS and 2 mM L-glutamine. Recombinant human E-cadherin Fc chimera (rhEad/Fc) and mouse E-cadherin Fc chimera (rmEcad/Fc) (sEcad), purchased from R&D Systems, represents the cDNA sequence encoding the extracellular domain of human E-cadherin (amino acid residues 155-707 or EC1-EC5 domains of the E-cadherin extracellular domain) fused by means of a polypeptide linker to the Fc region of human IgG1 that is 6× histidine-tagged at the C-terminus.

Animal Studies.

Wild-type (WT) and MMTV-PyMT mouse breeders were obtained from Jackson Laboratories and mated according to vendor's specifications. At 90-days of age, tumors, blood and urine were collected and stored until assayed. Overnight urine collections were obtained from wild-type (WT) and 90-day old MMTV-PyMT transgenic mice using specialized metabolic cages (Tecniplast USA Inc.) with food and water supplied ad libitum. For the chronic photocarcinogenesis model, the dorsal skin of SKH-1 mice (Charles River Laboratories) were exposed to 180 mJ/cm² UVB twice per week for 35 weeks, as previously described (Kim et al., Photochem Photobiol. 75: 58-67, 2002). All experimental procedures were approved by the Institutional Laboratory Animal Care and Use Committee at SBU.

Patients and Tissues.

Human HER2 breast, TNBC, skin SCC and head & neck cancer specimens were obtained from the NCI Cooperative Human Tissue Network (CHTN) and from Proteogenex (Culver City, Calif.). The use of human tissues was approved by the Institutional Review Board at Stony Brook University.

Immunoblotting.

These methods are as previously described.

ELISA Assay.

Levels of sEcad in the serum and urine of MMTV-PyMT mice, or conditioned media of serum starved cells, were quantified using human E-cadherin Quantikine ELISA Kits (R&D Systems), according to the manufacturer's recommendations. Serum, urine and conditioned media were diluted appropriately to fall within the standard range of the assay. Urinary sEcad results were corrected for urine creatinine concentrations using the QuantiChrom Creatinine Assay Kit (DICT-500, BioAssay System. Each experiment was performed in triplicate.

Semi-Quantitative PCR:

Total cellular RNA from normal curetted mouse epidermis, resected papillomas and SCCs (N=2) were extracted and the level of E-cadherin mRNA was determined using a semi-quantitative RT-PCR recation as described in Kyrkanides et al. (J. Neuroimmunol. 188:39-47, 2007) using the following mouse E-cadherin primers:

upper primer
(SEQ ID NO: 2)
5′-GGACTACGATTATCTGAACG-3′,
lower primer
(SEQ ID NO: 3)
5′-AACACACACACTATCCAGC-3′.

### Example 4: Soluble E-Cadherin Interacts and Activates Receptor Tyrosine Kinases, MAPK, and P13K/Akt/mTOR Signaling

Because the human epidermal growth factor receptor (HER) family regulates diverse biological responses, including proliferation, cell motility and tumor cell survival (Yarden and Siliwkowski, Nature Reviews. 2:127-137, 2001), we used immunoprecipitation and western blot assays to examine whether sEcad may promote carcinogenesis by interacting with this family of receptors. Extracts prepared from human ductal adenocarcinomas or resected MMTV-PyMT tumors were immunoprecipitated with HER1, HER2 or HER3-specific antibodies, and immunoblotted with an ectodomain-specific E-cadherin antibody. HER1, HER2 and HER3 associated with sEcad in both human and mouse breast tumors, and immunofluorescence analyses showed co-localization of HER1 and HER2 with this domain in resected MMTV-PyMT tumors, consistent with previous studies showing HER2-sEcad complexes in SKBR3 cells (Najy et al., J. Biol. Chem. 283:18393-401, 2008). In human TNBC tumors, which predominately express HER1, we found sEcad-HER1 interactions, but no association with the 120 kDa intact FL-Ecad protein. In human skin (Brouxhon et al., Oncogene 2012; In press) and head & neck SCC specimens, we also found association of endogenous sEcad with HER1, HER2 and IGF-1R, whereas no appreciable interaction of FL-Ecad with the above receptors was noted.

The association of sEcad with these RTK was also confirmed by in vitro studies. Specifically, in cell culture systems, extracts from sEcad (rhEcad/Fc:His tagged) treated E-cadherin positive MCF-7 and E-cadherin negative SKBR3 and MDA-MB-231 breast cancer cells were immunoprecipitated with HER1-4-specific antibodies and analyzed by western blotting with a His-tagged-specific antibody. Here, we detected HER1-4-sEcad, HER1-2-sEcad and HER1-sEcad interactions in MCF, SKBR3 and MDA-MB-231 breast cancer cells, respectively. Similarly, sEcad-HER1, sEcad-HER2 and sEcad-IGF-1R interactions were found in sEcad-stimulated skin PAM212 cells (Brouxhon et al., Oncogene 2012; In press) and sEcad-HER1 and sEcad-IGF-1R interactions in FADU oropharyngeal SCCs. Because ligand binding to the HER family of receptors induces the formation of homo- and heterodimers, resulting in phosphorylation on specific tyrosine residues within the cytoplasmic domain (Yarden and Siliwkowski, Nature Reviews. 2:127-137, 2001), we next used western blot assays to examine whether sEcad promotes HER phosphorylation and downstream signaling. In breast cancer cells, HER1-4 phosphorylation was notably increased by sEcad (rhEcad/Fc) in both the E-cadherin positive MCF-7 and negative SKBR3 cells 26 h after stimulation, consistent with a report demonstrating that sEcad-HER2 complexes result in HER2-HER3 heterodimerization, and HER3 and ERK1/2 phosphorylation in SKBR3 cells (Najy et al., J. Biol. Chem. 283:18393-401, 2008). In MDA-MB-231 TNBC cells, which predominately express HER1, sEcad induced HER1 phosphorylation. In skin and oropharyngeal SCC cell lines, HER1, HER2 and IGF-1 receptor phosphorylation and HER1 and IGF-1R phosphorylation was notably increased by sEcad administration, respectively. In separate experiments, the more invasive CC4A skin SCC cell line expressed IGF-1R and HER2 (p95), which were also dose dependently activated after sEcad treatment (Brouxhon et al., Oncogene 2012; In press). Lastly, using human and mouse phospho-RTK antibody array kits (R&D Systems), we validate that sEcad activates HER1-4, as well as other RTK including, but not limited to, the RET, FGFR, TIE, Axl, Eph, VEGF and MuSK family of receptors in MCF-7 and PAM212 cells.

We speculated that this effect may be mediated by molecular abnormalities in signaling pathways that promote cell growth downstream of HERs. Specifically, the lipid kinase phosphoinositide-3-kinase (PI3K) and the protein-serine/threonine kinases Akt, and mTOR signaling pathways are important mediators of HER receptor signaling and are causally involved in breast and other malignancies (Schmelzle and Hall, Cell 103:253-262, 2000). In breast cancer cell lines, western blot analyses showed activation of ERK1/2, PI3K/Akt and mTOR in rhEcad/Fc stimulated MCF-7 and SKBR3 cells, and Akt in MDA-MB-231 TNBC cells. In the PAM212 and CC4A skin SCC cell lines, sEcad induced the phosphorylation of MEK1/2, ERK1/2, Akt and mTOR, but only CC4A cells also induced phospho-PI3K (Brouxhon et al., Oncogene 2012; In press). Along these lines, sEcad-treated FADU human oropharyngeal cells activated MEK1/2, ERK1/2 and PI3K/Akt phosphorylation. As the p70S6 kinase and eIF4E binding protein 1 (4EBP1) are among the most well characterized targets of mTOR (Schmelzle and Hall, Cell 103:253-262, 2000), we next studied the phosphorylation of p70S6K at Thr-389 and 4EBP1 at Thr-37 by western blotting. Not surprisingly, the phosphorylated p70S6K and 4EBP1 levels were higher in rhEcad/Fc treated MCF-7, SKBR3, PAM212, CC4A cells versus untreated controls.

As increasing evidence indicates that the PI3K/Akt pathway can regulate the IAP family of proteins (Asanuma et al., Cancer Res 65:11018-11025, 2005) and that IAP members are associated with de novo and acquired resistance (Oliveras-Ferraros et al., Biochem Biophys Res Commun 407:412-419, 2011), we next examined the effects of sEcad on the levels of IAPs in E-cadherin-negative and -positive cells using western blot analysis. To the best of our knowledge, our study is the first to show that exogenous sEcad upregulated many of the IAP family members, including survivin, cIAP-1, XIAP and livin in both MCF-7 and SKBR3 cells. However, in MDA-MB-231 cells only survivin and livin protein expression levels were increased in the presence of sEcad. These findings provide evidence that sEcad modulates IAP family members, although to varying degrees, in both HER2⁺ and HER2-TNBC cells that express or lack E-cadherin. Altogether, our results thus far suggest that sEcad promotes the survival and growth of cancers by up-regulating the HER and IGF-1R family of receptors and by activating downstream PI3K/Akt/mTOR and IAP pro-survival signaling.

The methods employed in the study described above included the following: Immunoprecipitation and Immunoblotting.

Immunoprecipitation assays were carried out by harvesting tissues or cells with immunoprecipitation lysis buffer (20 mM Tris-HCl, pH 7.5; 137 mM NaCl; 100 mM NaF; 10% glycerol (vol/vol); 1% (vol/vol) Nonidet P-40; 1 mM PMSF and protease inhibitor cocktail (Sigma)). After brief sonication, lysates were cleared by centrifugation at 4° C. Supernatants were precleared and incubated with EGFR/HER1, HER2, HER3, HER4 or E-cadherin ectodomain-specific antibodies for 4 h and protein A/G plus agarose beads (Santa Cruz, sc-2003) for 2 h at 4° C. The immunocomplexes were washed three times, boiled in sample buffer (60 mM Tris-Cl, pH 6.8; 2% SDS (vol/vol); 10% glycerol (vol/vol); 5% 3-mercaptoethanol (vol/vol); and 0.01% bromophenol blue (vol/vol), and loaded on SDS-PAGE for protein analysis. Protein extraction in cells was performed on ice using total protein extraction buffer: 20 mM Tris pH 7.5, 137 mM NaCl, 100 mM NaF, 10% glycerol, 1% NP40, 1 mM PMSF and protease inhibitor cocktail (Sigma). For normal and tumor tissues, sEcad was extracted in an aqueous solution (PBS) and not in the protein extraction buffer. Protein concentration was measured using a BCA Protein Assay Kit (Pierce). Protein samples (50-100 μg) were denatured at 95° C. and subsequently separated by 4-15% SDS-PAGE gel electrophoresis. After transfer to nitrocellulose membrane and blocking with 1% BSA, samples were probed with primary antibodies. Western blot images were captured using HP Scanjet G4050 and analyzed relative to G3PDH or actin using NIH Scion Image.

### Example 5: Soluble E-Cadherin Signals Though HERs and Acts Additively with the EGF Ligand to Promote Oncogenicity

As the HER family members are well known mediators of cell proliferation and migration, we next examined whether sEcad enhanced breast cancer proliferation, migration and formation of microtubules, stress fibers and focal adhesions in cell culture systems. In the MCF-7, SKBR3 and MDA-MB-231 breast cancer cell lines, BrdU incorporation was significantly increased with 10 and 20 μg/mL of sEcad (rhEcad/Fc), albeit to varying levels depending on the cell type. This result is in agreement with recent findings showing that exogenous sEcad increased proliferation in SKBR3 cells (Najy et al., J. Biol. Chem. 283:18393-401, 2008). A similar sEcad-induced increase in proliferation was noted in the oropharyngeal FADU and skin SCC (PAM212, CC4A, SCC13 and SCC12b) cell lines. The effects of sEcad on migration and invasion were then examined in vitro. Using Transwell plates, sEcad treatment of breast (MCF-7, SKBR3), skin (PAM212, CC4A, SCC12b, SCC13) and oropharyngeal (FADU) cells induced a significant dose-dependent increase in migration compared to untreated controls. As cytoskeletal rearrangement and formation of newly branched F-actin and microtubules have been suggested to play a role in regulating cell migration (Machesky. FEBS Letters 582:2102-2111, 2008), we next assessed actin polymerization and microtubule assembly in cells stimulated for 24 h with sEcad. A noticeable change in cell shape associated with lamellipodia and stress fibers, were noted in both sEcad treated MCF-7 and SKBR3 cells whereas serum-starved MDA-MB-231 cells displayed the presence of spindle-shaped cells. To assess whether sEcad promotes cell invasion, we assayed the ability of serum-starved cells treated with sEcad to invade through Transwell inserts coated with a thin layer of Matrigel. Breast (MDA-MB-231), skin (SCC12b, SCC13, PAM212 and CC4A) and the head & neck FADU cell line exhibited a dramatic increase in invasion through Matrigel in the presence of sEcad compared to untreated controls. Of note, baseline MCF-7 and SKBR3 cells typically exhibit minimal invasive characteristics. Because matrix metalloproteinases, particularly MMP-9 and MMP-2, are key players in tumor dissemination, we evaluated the levels of pro-and -active MMP-2 and MMP-9 in sEcad (rmEcad/Fc)-treated cutaneous SCC PAM212 cells. ELISA demonstrated a dose-dependent increase in MMP-9 secretion and gelatin zymography demonstrated an enhanced activation of MMP-9 and MMP-2 in the conditioned media of these treated cells (Brouxhon et al., Oncogene 2012; In press). To confirm that sEcad enhances invasion via MMPs, we examined the invasive potential of PAM212 cells treated with 20 μg/mL rmEcad/Fc in the presence or absence of GM6001 (Ilomastat; BIOMOL, Plymouth, Pa.), a potent MMP inhibitor that reduces the activity of collagenases and gelatinases, including MMP-2 and MMP-9. sEcad-stimulated invasion was completely blocked in the presence of GM6001, providing further evidence for the role of sEcad-induced MMP activity in skin cancer invasion (Brouxhon et al., Oncogene 2012; In press). Altogether, these data suggest that sEcad in the tumor microenvironment acts in an autocrine and/or paracrine manner to facilitate tumor cell proliferation, migration, invasion and the production of pro-invasive MMPs even in tumor cells that lack E-cadherin.

To validate that sEcad signals via the HER family of receptors to induce these functional effects, we next treated different types of cancer cells with various HER inhibitors in the presence or absence of sEcad (rmEcad/Fc or rhEcad/Fc) and evaluated proliferation, migration and invasion. In breast cancer cells, the pan HER inhibitor Canertinib (1 μM), the HER1/HER2 inhibitor Lapatinib (5 μM), and the HER1 inhibitor Gefitinib (5 μM) significantly reduced both basal and sEcad-induced BrdU incorporation. Moreover, Canertinib, Lapatinib and Gefitinib exhibited a similar decrease on basal and rhEcad/Fc-mediated migration, whereas Mubritinib only blocked sECad-induced MCF-7 migration. Next, in an effort to evaluate breast cancer invasion, we used the highly aggressive MDA-MB-231 cell line that exhibits a high invasion capacity and overexpresses HER1, but minimal levels of HER2, 3 and 4 (Sahin et al., BMC Syst Biol. 3:1-20, 2009). In the presence of Gefitinib, both basal and sEcad-induced invasion was significantly reduced compared to untreated controls. Next, we evaluated the effects of Canertinib, Lapatinib and Gefitinib, in the presence or absence of sEcad, on the mitogenic, migratory and invasive capabilities in PAM212 skin SCC cells (Brouxhon et al., Oncogene 2012; In press). Interestingly, all three inhibitors effectively inhibited sEcad-induced proliferation, migration and invasion. Notably, in the PAM212 cells, the pan HER inhibitor Canertinib completely abrogated sEcad-induced HER1 and HER2 phosphorylation, whereas Lapatinib significantly down-regulated the sEcad-induced phosphorylation of HER1, ERK1/2, pAkt and p70S6K, but exhibited minimal changes on pHER2, PI3K and 4EBP1. Gefitinib decreased the sEcad-induced phosphorylation of HER1, ERK1/2, Akt, p70S6K and PI3K, but had only minimal effects on pHER2 and p4EBP1. Canertinib, Lapatinib and Gefitinib, in the presence of sEcad, did not alter phospho-IGF-1R levels compared to their respective corresponding controls.

To understand whether activation of MEK and PI3K are both necessary for sEcad-mediated cell proliferation, migration and invasion, the PI3K inhibitor (LY294002) and MEK inhibitor (PD98059) were used either alone or in combination in PAM212 cells (Brouxhon et al., Oncogene 2012; In press). Both ERK1/2 and Akt/p70S6K phosphorylation were decreased after PI3K inhibition. Similarly, MEK inhibition resulted in decreased ERK1/2 phosphorylation. Interestingly, mTOR/p70S6K/4EBP1 phosphorylation increased in the presence of the MEK inhibitor PD98059, even in the absence of rmEcad/Fc. This is not surprising, since it has become increasingly apparent that a complex network of horizontal and vertical signaling crosstalk and feedback loops exist between the MAPK axis and the mTOR axis, whereby inhibition of components of one axis results in a compensatory upregulation of the alternate pathway (Higgins and Baselga, J. Clin. Invest. 121: 3797-3803, 2011). This may explain why Akt phosphorylation was enhanced by sEcad in the presence of the MEK inhibitor. In addition, since we have shown that sEcad can activate both HER and IGF-1R family members, it is plausible that this sEcad-induced Akt/mTOR/p70S6K/4EBP1 activation may occur via HER1-4 or IGF-1R signaling. Functionally, our data further show significant reductions in sEcad-induced PAM212 proliferation in the presence of the PI3K inhibitor LY294002, but less significant effects with MEK inhibitor PD98059 (Brouxhon et al., Oncogene 2012; In press). Because the mTOR/p70S6K/4EBP1 signaling branch plays a central role in regulating cell growth and proliferation (Bjornsti and Houghton, Nat Rev Cancer. 4: 335-348, 2004), it is feasible that the enhanced sEcad-induced mTOR/p70 S6-kinase/4EBP1 expression levels seen with PD98059 may mediate these effects. Furthermore, our data suggest that sEcad-induced proliferation, migration and invasion are PI3K and MEK-dependent, since irrespective of which inhibitor is utilized, these functional effects are blocked. This conclusion is supported by our results, where the PI3K and MEK inhibitors, either alone or in combination, significantly inhibited sEcad-induced activation of MMP-2 and MMP-9. Altogether, these results provide evidence that sEcad acts via the PI3K and MEK pathways and its substrates Akt/mTOR and ERK1/2 to facilitate pro-oncogenic properties in skin cancer cells.

Because the tumor mileu likely contains HER-related ligands that could potentially compete, act additively or synergize with sEcad for binding, we next investigated whether exogenous ligands such as EGF could alter the pro-oncogenic effects induced by sEcad. Here, MCF-7 breast cancer cells were treated for 24 h with equimolar concentrations of EGF, sEcad alone, or sEcad in combination with EGF, and then proliferation, migration and invasion were assessed. Treatment of cell cultures with rhEGF or sEcad (rhEcad/Fc) alone, significantly increased proliferation, migration and invasion, but these pro-oncogenic effects were more potent with exogenous sEcad. Strikingly, combination treatment with equimolar rhEGF and sEcad resulted in a partial, but statistically significant, additive effect on proliferation, migration and invasion compared to sEcad or EGF treatments alone. Next, in order to examine HER and downstream signaling, we evaluated this axis in the presence or absence of equimolar concentrations of sEcad, rhEGF, and sEcad and rhEGF in combination. As anticipated, rhEGF alone exhibited more potent effects on HER1 and ERK1/2 phosphorylation compared to sEcad alone. In contrast, exogenous sEcad demonstrated enhanced phospho-HER3, Akt and less of an increase on phospho-mTOR expression levels. In combination, no significant additive or synergistic effects were noted. Together, these data provide evidence that sEcad signals via HERs and acts additively with EGF to promote breast cancer oncogenicity.

The methods employed in the study described above included the following:

Cell Lines and Reagents.

Human MCF-7, SKBR3 and MD-MB-231 cells were purchased from ATCC, and cultured according to ATCC recommendations. PAM212 and CC4A cells were cultured, as previously described. SCC12b and SCC13 cells were kindly provided by Marcia Simon (Stony brook University, Stony Brook, N.Y.). Recombinant human and mouse E-cadherin Fc chimera (sEcad; rhEad/Fc and rmEcad/Fc) was purchased from R&D Systems. Canertinib, Lapatinib, Gefitinib and Mubritinib were purchased from LC Laboratories (Woburn, Mass.). PI3K (LY294002, 20 μM) and MEK (PD98059, 20 μM) inhibitors were purchased from EMD (Billerica, Mass.). GM6001 was purchased from Biomol (Ann Arbor, Mich.). Recombinant human EGF (rhEGF) was purchased from Enzo Life Sciences (Farmingdale, N.Y.).

Gelatin Zymography.

Conditioned media from control or sEcad treated cells were concentrated 10-fold using Centricon devices (Millipore; Bedford, Mass.) according to the manufacturer's instructions and applied to SDS-PAGE gels containing gelatin (0.1% w/v). Gels were washed twice in renaturing buffer (Invitrogen) and incubated at 37° C. for 24 h in developing buffer (Invitrogen). Gels were stained with Coomassie blue and destained with methanol:acetic acid (50:10). Zymogram is representative of triplicate experiments.

ELISA Assays.

Conditioned media from human and mouse cells were analyzed for MMP-9 using human and mouse E-cadherin Quantikine ELISA Kits and mouse MMP-9 ELISA kit (R&D Systems, Minneapolis, Minn.), according to the manufacturer's specifications, respectively. For measuring mouse MMP-9, serum-free medium was added to cell cultures and cells were exposed to increasing concentrations of sEcad (rmEcad/Fc; R&D Systems) for 24 hours. The culture media were collected and concentrated using Centrifugal Ultra Filters (Millipore, Billerica, Mass.) and MMP-9 levels were measured by ELISA. Each experiment was performed in triplicate.

Western Blot Analyses.

Lystes from cells were processed for western blots as described previously (Brouxhon et al., Cancer Res. 67:7654-64, 2007). Antibodies against total HER1 and HER 4 and phosphorylated HER1-4, pPI3K, pAkt, pmTOR, p4E-BP1, pRaptor and pp70S6K obtained from Cell Signaling Technologies. Monoclonal antibodies to HER3 and HER4 were obtained from Thermo Scientific. Polyclonal antibody to the extracellular domain of E-cadherin and monoclonal antibody to α-actin were obtained from Santa Cruz Biotechnology. Monoclonal antibody to the extracellular domain of E-cadherin (DECMA-1) and to His Tag were obtained from Sigma and Abcam, respectively. Monoclonal antibody to G3PDH, ubiquitin and rat IgG1 were obtained from Ambion, Zymed and Southern Biotechnology, respectively.

Immunofluorescence.

Cells cultured on chamber slides (Nalge Nunc International) were fixed with 4% formaldehyde for 10 minutes and blocked for 20 minutes in PBS containing 1% (wt/vol) BSA and incubated in HER1-4 antibodies, as previously described (Brouxhon et al., Cancer Res. 67:7654-64, 2007). For staining of actin, microtubules and double labeling, cells were fixed with 100% (vol/vol) methanol at −20° C. for 15 minutes and blocked in 1% (wt/vol) BSA-0.4% (vol/vol) Triton X-100 for 20 minutes. Cells were incubated with Alexa 594-conjugated phalloidin (Invitrogen) or with monoclonal anti-α-tubulin (Invitrogen) overnight at 4° C., followed by incubation with corresponding fluorescence-conjugated secondary antibodies (Invitrogen). Nuclei were counterstained with 2 μg/ml Hoechst 33342 (Invitrogen).

Cell Proliferation, Migration and Invasion Assays.

For BrdU staining, cells were incubated with 10 μM BrdU for 2 h prior to fixation and processed for immunofluorescence microscopy as previously described (Brouxhon et al., Cancer Res. 67:7654-64, 2007). For BrdU analyses, cells were counted from 6 low-power fields (×100) per section. BrdU incorporation was also analyzed by a cell proliferation ELISA 5-bromo-2′-deoxyuridine (BrdU) (colorimetric) kit (Roche, Stockholm, Sweden), according to the manufacturer's instructions. Migration and invasion of cells were measured using 8.0 μm pore BD BioCoat Control Insert 24-well plates (no. 354578) and Matrigel Invasion Chamber 24-well Plates (no. 354480), respectively (BD Bioscience). Cells were collected, washed, and 2×10⁵ cells were plated in 0.4% FBS medium in the top chamber, with 0-20 μg/mL of rhEcad/Fc in the bottom chamber. After 22 h, cells on the top were removed using a cotton swab. Migrated or invaded cells on the lower surface were fixed with methanol, stained with 0.5% crystal violet, examined by bright field microscopy and photographed. Migration or invasion was quantitated by counting migrated or invaded cells in at least ten random high-power fields per insert and expressed as averages. Results are presented as fold change of the number of migrated/invaded cells to the untreated controls in triplicate experiments.

Statistical Analysis.

Data are presented as means with standard errors (SEM). Independent two-sample t-tests compare differences between two groups. A p-value below 0.05 indicated statistical significance. Statistics were analyzed with SPSS 15.0 software (SPSS Inc, Chicago, Ill., USA).

1-24. (canceled)

25. A pharmaceutical composition comprising a therapeutically effective amount of a first agent and a second agent, wherein:
the first agent, is an antibody or a biologically active fragment thereof that specifically binds to the Ectodomain of E-cadherin or the soluble E cadherin (sEcad) fragment thereof, without binding the first subdomain (EC1) of E-cadherin or sEcad;
the second agent is a small molecule, an antibody, or a biologically active fragment thereof that inhibits one or more of HER1, with the proviso that cetuximab and panitumumab are excluded; HER2, with the proviso that trastuzumab is excluded;
HER3; and HER4.

26. The pharmaceutical composition of claim 25, wherein the second agent is an antibody that inhibits HER2.

27. The pharmaceutical composition of claim 25, wherein the second agent is selected from the group consisting of gefitinib and erlotinib, lapatinib, mubritinib, pertuzumab and canertinib.

28. The pharmaceutical composition of claim 25, wherein the composition is delivered in a pharmaceutical formulation that produces, upon administration to a patient, a serum level of the first agent of about 1-50 mg/kg.

29. A method of treating cancer, the method comprising administering to a patient in need thereof:
an antibody or a biologically active fragment thereof that specifically binds to the Ectodomain of E-cadherin or the soluble E cadherin (sEcad) fragment thereof, without binding the first subdomain (EC1) of E-cadherin or sEcad; and
a small molecule, an antibody, or a biologically active fragment thereof that inhibits one or more of HER1, with the proviso that cetuximab and panitumumab are excluded; inhibits HER2, with the proviso that trastuzumab is excluded: inhibits HER3; or inhibits HER4.

30. The method of claim 29, wherein the cancer is an epithelial cancer.

31. The method of claim 29, wherein the cancer is a cancer of the alimentary canal, central nervous system, breast, skin, reproductive system, lung, or urinary tract.

32. The method of claim 31, wherein the cancer of the alimentary canal is a cancer of the mouth, throat, esophagus, stomach, intestine, colon, rectum or anus.

33. The method of claim 31, wherein the cancer of the skin is squamous cell carcinoma or melanoma.

34. The method of claim 31, wherein the cancer of the reproductive system is cervical cancer, uterine cancer, ovarian cancer, vulval or labial cancer, prostate cancer, testicular cancer, or cancer of the male genital tract.

35. The method of claim 29, wherein the first and second agent are administered in two separate dosage forms.

36. The method of claim 29, wherein the second agent is an antibody that inhibits HER2.

37. The method of claim 29, wherein the second agent is selected from the group consisting of gefitinib and erlotinib, lapatinib, mubritinib, pertuzumab and canertinib.

38. The method of claim 29, further comprising the step of providing a biological sample from the patient and determining whether the sample includes an elevated level of sEcad.


SEQUENCE LISTING (5 sequences)

SEQ ID NO: 1 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 2 - DNA, 20 bp, Artificial Sequence
  Description of Artificial Sequence Synthetic primer
ggactacgattatctgaacg

SEQ ID NO: 3 - DNA, 19 bp, Artificial Sequence
  Description of Artificial Sequence Synthetic primer
aacacacacactatccagc

SEQ ID NO: 4 - protein, 15 aa, Artificial Sequence
  Description of Artificial Sequence Synthetic peptide
GGGGSGGGGSGGGGS

SEQ ID NO: 5 - protein, 6 aa, Artificial Sequence
  Description of Artificial Sequence Synthetic 6xHis tag
HHHHHH